(19) United States
(12) Patent Application Publication
(10) Pub. No.: US 2022/0275274 A1
(43) Pub. Date: Sep. 1, 2022

(54) NEUTRAL FLUORESCENT MITOCHONDRIAL MARKER BASED ON NITROGEN-CONTAINING HETEROCYCLE, PREPARATION METHOD AND USE THEREOF

(71) Applicant: SOOCHOW UNIVERSITY, Suzhou (CN)

(72) Inventors: Jianfeng GE, Suzhou (CN); Yue WANG, Suzhou (CN); Ru SUN, Suzhou (CN)

(21) Appl. No.: 17/631,432

(22) PCT Filed: Feb. 4, 2021
(86) PCT No.: PCT/CN2021/075160

§ 371 Date: Jan. 28, 2022

(30) Foreign Application Priority Data
Feb. 25, 2020 (CN) 202010117779.0

(51) Int. Cl. C09K 11/06; G01N 21/64; C07F 5/02

(52) U.S. Cl. CPC C09K 11/06; G01N 21/6402; C07F 5/022; C09K 2211/1033; C09K 2211/1044; C09K 2211/1059; C09K 2211/1088

(57) ABSTRACT

The present invention provides a neutral fluorescent mitochondrial marker based on a nitrogen-containing heterocycle, and a preparation method and use thereof. The fluorophore in present invention is a heterocycle having a N—H bond and targeting mitochondria, which solves the problem that the ability of a fluorescent dye with a neutral structure to target organelles is random and uncertain, and also avoids the problem that the neutral fluorophore is a commercial marker for lipid droplets in cells. In the present invention, the organelle targeting ability of an original fluorophore is regulated by creatively modifying its structure while the optical performance of the fluorophore is improved. The marker improves the biological properties of the fluorophore, and the nitrogen-containing heterocycle building block is cheap and readily available, which is beneficial to controlling the cost of the new dye. The present invention has great scientific significance and commercial value.

### FIELD OF THE INVENTION

The present invention relates to the fluorescent labeling technology, and more particularly to a novel neutral fluorescent mitochondrial marker based on a nitrogen-containing heterocycle.

### DESCRIPTION OF THE RELATED ART

Mitochondria are one of the most essential organelles in cells. In addition to being the main sites for aerobic respiration and providing energy for cells, mitochondria also participate in important physiological activities such as genetic material transfer and cell differentiation (see: Levenson, R.; Macara, I. G.; Smith, R. L.; Cantley, L.; Housman, D. Cell 1982, 28, 855.). Therefore, in scientific research, real-time monitoring of mitochondria is particularly important. Among various technical means, fluorescent labeling technology becomes notable due to its simple operation and low preparation cost. Various fluorescent probes and dyes targeting mitochondria are developed accordingly. Given the reported fluorescent probes and dyes targeting mitochondria, it can be easily found that they mosty have a triphenylphosphonium, a pyridinium and an indolium in their main structures (see Angew CHem Int Ed 2016, 55, 13658.). This is true even for the most commonly used commercial red and green mitochondrial markers. This is because the presence of a proton pump on the inner mitochondrial membrane makes it easier for these cationic dyes to penetrate the mitochondrial membrane and accumulate in the mitochondria. However, they are accompanied by problems that these cations will change the mitochondrial membrane potential after entering the mitochondria, leading to cell apoptosis (see: Sens Actuators B 2019, 292, 16.).

### SUMMARY OF THE INVENTION

The present invention provides a novel neutral fluorescent mitochondrial marker based on a nitrogen-containing heterocycle, which can be used as a fluorescent mitochondrial marker. The present invention solves the problem that the ability of a fluorescent dye with a neutral structure targeting organelles is random and uncertain, and also avoids the problem that the neutral fluorophore is a commercial marker for lipid droplets in cells. In the present invention, the organelle targeting ability of an original fluorophore can be regulated by creatively modifying its structure while the optical performance of the fluorophore is improved. The marker improves the biological properties of the fluorophore, and the nitrogen-containing heterocycle building block is cheap and readily available, which is beneficial to controlling the cost of the new dye.

The following technical solutions are adopted in the present invention.

A neutral fluorescent mitochondrial marker based on a nitrogen-containing heterocycle has a structure represented by one of the following chemical formulas:

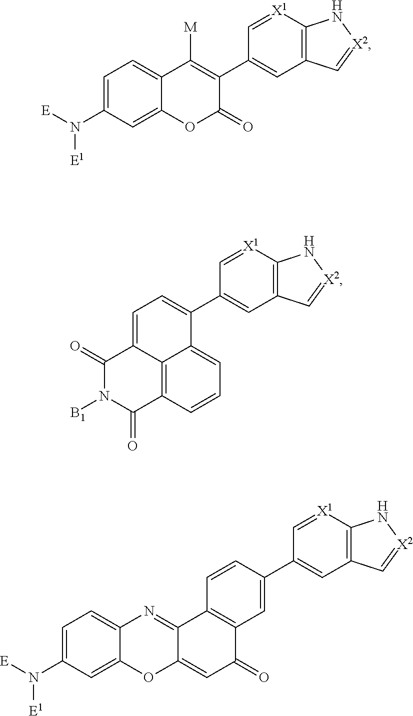

where X¹ and X² are independently selected from CH or heteroatoms; and M, E, E¹, and B₁ are independently selected from an alkyl group with less than 6 carbon atoms. The neutral fluorescent mitochondrial marker based on a nitrogen-containing heterocycle of the present invention has a N—H bond.

Preferably, the neutral fluorescent mitochondrial marker based on a nitrogen-containing heterocycle has a structure represented by one of the following chemical formulas:

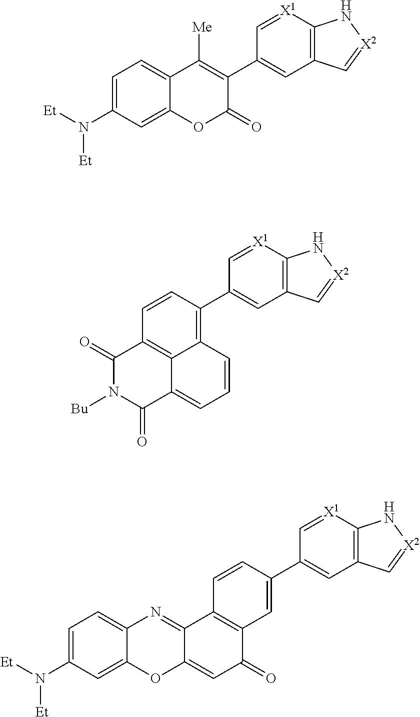

where X¹ is selected from CH or N; and X² is selected from CH or N.

The present invention provides use of the neutral fluorescent mitochondrial marker based on a nitrogen-containing heterocycle in fluorescent labeling of mitochondria; or use of the neutral fluorescent mitochondrial marker based on a nitrogen-containing heterocycle in the preparation of a fluorescent mitochondrial labeling reagent.

The present invention provides a method for preparing a neutral fluorescent mitochondrial marker based on a nitrogen-containing heterocycle, which is one of the following preparation methods:

(1) reacting a compound 6 with a compound 7 to obtain a compound 8, and deprotecting the compound 8, to obtain a neutral fluorescent mitochondrial marker based on a nitrogen-containing heterocycle; or

(2) reacting a compound 9 with a compound 7 to obtain a compound 10, and deprotecting the compound 10, to obtain a neutral fluorescent mitochondrial marker based on a nitrogen-containing heterocycle; or

(3) reacting a compound 13 with a compound 7 to obtain a compound 14, and deprotecting the compound 14, to obtain a neutral fluorescent mitochondrial marker based on a nitrogen-containing heterocycle.

The present invention provides a cell imaging method, including the following steps:

(1) reacting a compound 6 with a compound 7 to obtain a compound 8, and deprotecting the compound 8, to obtain a neutral fluorescent mitochondrial marker based on a nitrogen-containing heterocycle; or

(2) reacting a compound 9 with a compound 7 to obtain a compound 10, and deprotecting the compound 10, to obtain a neutral fluorescent mitochondrial marker based on a nitrogen-containing heterocycle; or

(3) reacting a compound 13 with a compound 7 to obtain a compound 14, and deprotecting the compound 14, to obtain a neutral fluorescent mitochondrial marker based on a nitrogen-containing heterocycle; and

(4) co-incubating the neutral fluorescent mitochondrial marker based on a nitrogen-containing heterocycle prepared in Step (1) or Step (2) with the cells, adding a red mitochondrial marker, and imaging the cells after continuous incubation; or

co-incubating the neutral fluorescent mitochondrial marker based on a nitrogen-containing heterocycle prepared in Step (3) with the cells, adding a green mitochondrial marker, and imaging the cells after continuous incubation. The cells include normal cells and cancer cells.

In the present invention, the deprotection is carried out in the presence of hydrochloric acid; the reaction of the compound 6 is reacted with the compound 7 in the presence of a noble metal salt catalyst, preferably under an alkaline condition; the compound 9 is reacted with the compound 7 in the presence of a noble metal salt catalyst, preferably under an alkaline condition; and the compound 13 is reacted with the compound 7 in the presence of a noble metal salt catalyst, preferably under an alkaline condition. More preferably, the noble metal salt catalyst includes a palladium salt catalyst.

In the present invention, the compounds have the chemical structural formulas as shown below:

| | |
|---|---|
| 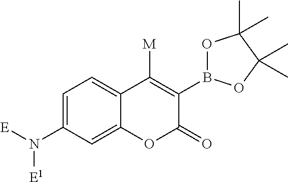 | compound 6 |
| 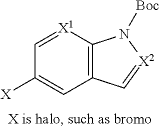 | compound 7 |
| 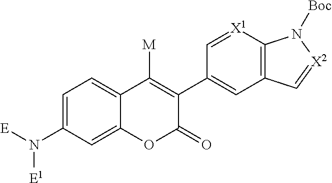 | compound 8 |
| 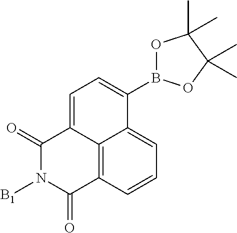 | compound 9 |
| 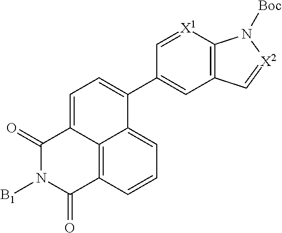 | compound 10 |
| 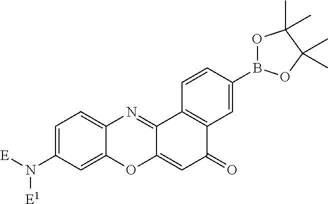 | compound 13 |

The compound 14 has a chemical structural formula below:

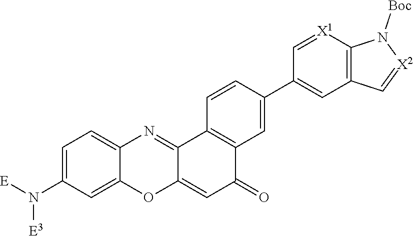

where the heterocycles have a N—H bond, X¹ and X² are independently selected from CH or heteroatoms; and M, E, E¹, and B₁ are a substituent independently selected from an alkyl group with less than 6 carbon atoms. The alkyl group in the present invention means a saturated branched or straight monovalent hydrocarbon group with 1 to 6 carbon atoms, such as methyl (Me), n-butyl (Bu), ethyl (Et) and the like.

In the present invention, the cells are imaged under a laser confocal microscope; in the blue channel, light of 405 nm is used for excitation, and a fluorescence signal in the range of 410-500 nm is collected; in the red channel, light of 561 nm is used for excitation, and a fluorescence signal in the range of 570-750 nm is collected; and in the green channel, light of 488 is used for excitation, and a fluorescence signal in the range of 500-550 nm is collected.

The present invention provides a neutral fluorescent mitochondrial marker based on a nitrogen-containing heterocycle for the first time, which enables cell imaging after co-incubation with the cells. In the present invention, the organelle targeting ability of an original fluorophore is regulated by creative modification of its structure while the optical performance of the fluorophore is improved. The marker has low cytotoxicity during cell imaging, has little damage to biological samples, and is not affected by other organelles. By using the marker, the cell sample can be observed for a long time. The marker improves the biological properties of the fluorophore, and the nitrogen-containing heterocycle building block is cheap and readily available, which is beneficial to controlling the cost of the new dye.

### BRIEF DESCRIPTION OF THE DRAWINGS

FIG. 1 shows a synthesis route of a dye according to the present invention;

FIG. 2 is a ¹H NMR spectrum of dye 1a;

FIG. 3 shows the ultraviolet-visible absorption spectrum and fluorescence spectrum of dye 1a in chloroform;

FIG. 4 shows the ultraviolet-visible absorption spectrum and fluorescence spectrum of dye 1b in chloroform;

FIG. 5 shows the ultraviolet-visible absorption spectrum and fluorescence spectrum of dye 1c in chloroform;

FIG. 6 shows the ultraviolet-visible absorption spectrum and fluorescence spectrum of dye 2a in chloroform;

FIG. 7 shows the ultraviolet-visible absorption spectrum and fluorescence spectrum of dye 2b in chloroform;

FIG. 8 shows the ultraviolet-visible absorption spectrum and fluorescence spectrum of dye 2c in chloroform;

FIG. 9 shows the ultraviolet-visible absorption spectrum and fluorescence spectrum of dye 3a in chloroform;

FIG. 10 shows the ultraviolet-visible absorption spectrum and fluorescence spectrum of dye 3b in chloroform;

FIG. 11 shows the ultraviolet-visible absorption spectrum and fluorescence spectrum of dye 3c in chloroform;

FIG. 12 shows the ultraviolet-visible absorption spectrum and fluorescence spectrum of dye 3d in chloroform;

FIG. 13 shows the ultraviolet-visible absorption spectrum and fluorescence spectrum of dye 4 in chloroform;

FIG. 14 is a cell image with dye 1a in L929 cells and HeLa cells;

FIG. 15 is a cell image with dye 1b in L929 cells and HeLa cells;

FIG. 16 is a cell image with dye 1c in L929 cells and HeLa cells;

FIG. 17 is a cell image with dye 2a in L929 cells and HeLa cells;

FIG. 18 is a cell image with dye 2b in L929 cells and HeLa cells;

FIG. 19 is a cell image with dye 2c in L929 cells and HeLa cells;

FIG. 20 is a cell image with dye 3a in L929 cells and HeLa cells;

FIG. 21 is a cell image with dye 3b in L929 cells and HeLa cells;

FIG. 22 is a cell image with dye 3c in L929 cells and HeLa cells;

FIG. 23 is a cell image with dye 3d in L929 cells;

FIG. 24 is a cell image with dye 3d in HeLa cells; and

FIG. 25 is a cell image with dye 4 in HeLa cells.

### DETAILED DESCRIPTION OF THE PREFERRED EMBODIMENTS

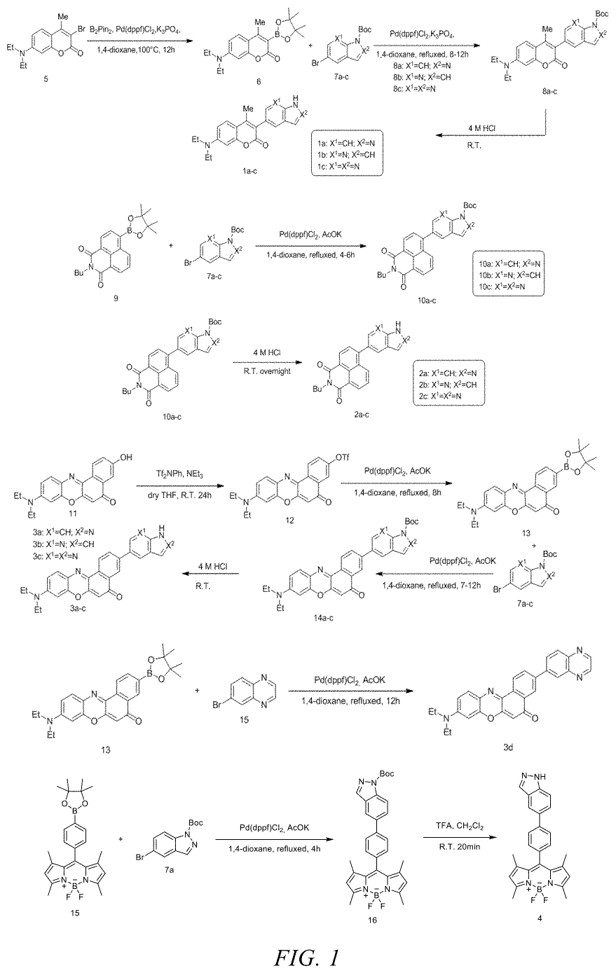

The synthesis route in the examples of the present invention is shown in FIG. 1, where the number below the chemical formula represents the compound. In the synthesis of the compound of the present invention, the ratios of raw materials and the purification methods are conventional ratios or conventional purification methods. The examples are illustrative.

### EXAMPLES

The compound 5 (2.0 mmol, 618.1 mg), bis(pinacolato)diboron (2.5 mmol, 634.8 mg), [1,1′-bis(diphenylphosphino)ferrocene]palladium dichloride (0.2 mmol, 146.3 mg), and potassium phosphate (4.0 mg, 849.1 mg) were dissolved in 1,4-dioxane (25.0 ml). The reaction system was purged three times with nitrogen, and reacted for 12 hrs at 100° C. After cooling down to room temperature, the reaction mixture was suction filtered, and the solvent in the filtrate was removed by rotary evaporation. The residue was separated by column chromatography (eluant: petroleum ether/ethyl acetate (5/1, v/v)) to obtain a light yellow immediate 6 (244.9 mg, yield 35%). NMR test: (400 MHz, DMSO-d₆) ¹H NMR (400 MHz, DMSO-d₆) δ(ppm) 7.52 (d, 1H, J=9.0, Ar—H), 6.67 (d, 1H, J=8.9, Ar—H), 6.48 (s, 1H, Ar—H), 3.43 (q, J=6.9 Hz, 4H, 2×CH₂), 2.37 (s, 3H, CH₃), 1.30 (s, 12H, 4×CH₃), 1.12 (t, J=6.1 Hz, 6H, 2×CH₃); (151 MHz, CDCl₃) ¹³C NMR (151 MHz, CDCl₃) δ(ppm) 163.4, 159.1, 156.4, 150.8, 125.9, 109.5, 108.1, 97.4, 84.0, 44.7, 24.8, 18.0, 12.5.

The immediate 6 (1.0 mmol, 357.2 mg), compound 7a (t-butyl 5-bromo-1H-indazol -1-carboxylate, 1.2 mmol, 355.2 mg), [1,1′-bis(diphenylphosphino)-ferrocene]palladium dichloride (0.1 mmol, 73.1 mg), and potassium phosphate (2.0 mmol, 424.5 mg) were dissolved in 1,4-dioxane (15.0 mL). The reaction system was purged three times with nitrogen, and then reacted for 12 hrs under reflux. After cooling down to room temperature, the reaction mixture was suction filtered, and the solvent in the filtrate was removed by rotary evaporation. The residue was separated by column chromatography (eluant: dichloromethane/methanol (100/1, v/v)) to obtain immediate 8a as a light yellow solid (192.3 mg, yield 43%). NMR spectra of immediate 8a: (400 MHz, CDCl₃) ¹H NMR (400 MHz, CDCl₃) δ(ppm) 8.23 (d, J=8.6 Hz, 1H, Ar—H), 8.19 (s, 1H, Ar—H), 7.68 (s, 1H, Ar—H), 7.48 (d, J=8.8 Hz, 2H, Ar—H), 6.64 (d, J=9.0 Hz, 2H, Ar—H), 6.57 (s, 1H, Ar—H), 3.44 (q, J=7.0 Hz, 4H, 2×CH₂), 1.74 (s, 9H, 3×CH₃) 2.24 (s, 3H, CH₃), 1.22 (t, J=6.0 Hz, 6H, 2×CH₃); (151 MHz, CDCl₃) ¹³C NMR (151 MHz, CDCl₃) δ(ppm) 162.2, 155.1, 150.4, 149.1, 148.8, 139.6, 139.0, 131.7, 131.0, 126.1, 126.0, 123.0, 120.2, 114.4, 109.4, 108.7, 97.5, 84.9, 44.8, 28.2, 24.8, 16.4, 12.4.

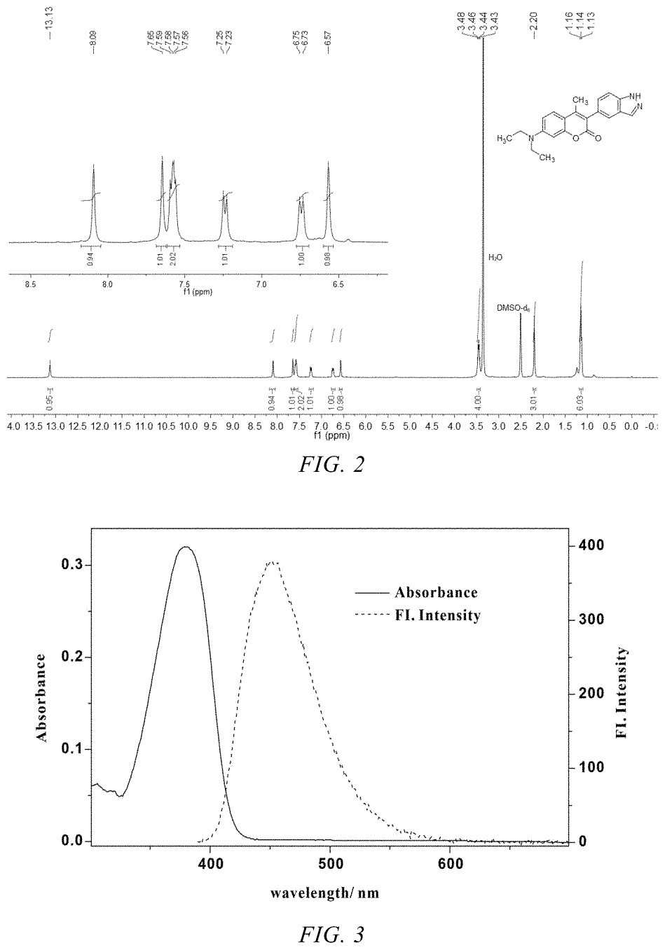

The immediate 8a (0.3 mmol, 134.2 mg) was dissolved in a mixed solution of concentrated hydrochloric acid (1.0 mL) and 1,4-dioxane (3.0 mL), and stirred at room temperature. The reaction was monitored by TLC. After the raw materials were completely reacted, a saturated sodium bicarbonate solution was added. The reaction solution was extracted with chloroform (3×30.0 mL). The organic layer was collected, dried over anhydrous Na₂SO₄, and evaporated to remove the solvent. The crude product was separated and purified by column chromatography (eluant: dichloromethane/methanol (30/1, v/v)), to obtain a pure product as a light yellow solid which was dye 1a (98.9 mg, yield 95%). FIG. 2 shows ¹H NMR spectrum of dye 1a (400 MHz, DMSO-d₆) ¹HNMR (400 MHz, DMSO-d₆) δ(ppm) 13.13 (s, 1H, N—H), 8.09 (s, 1H, Ar—H), 7.65 (s, 1H, Ar—H), 7.59 (d, J=5.3 Hz, 1H, Ar—H), 7.57 (d, J=4.9Hz, 1H, Ar—H), 7.24 (d, J=8.3 Hz, 1 H, Ar—H), 6.74 (d, J=8.5, 1H, Ar—H), 6.57 (s, 1H, Ar—H), 3.46 (q, J=7.3 Hz, 4H, 2×CH₂), 2.20 (s, 3H, CH₃), 1.14 (t, J=6.1 Hz, 6H, 2×CH₃); ¹³C NMR of dye 1a (151 MHz, CDCl₃) ¹³C NMR (151 MHz, CDCl₃) δ(ppm) 162.6, 155.1, 150.2, 148.8, 139.5, 135.0, 129.4, 128.0, 126.1, 123.3, 122.6, 121.1, 109.7, 109.5, 108.6, 97.5, 44.7, 16.4, 12.5.

The immediate 6 (1.0 mmol, 357.2 mg), compound 7b (t-butyl 5-bromo-1H-pyrrolo[2, 3-b]pyridin-1-carboxylate, 1.2 mmol, 355.2 mg), [1,1′-bis(diphenyl-phosphino) ferrocene]palladium dichloride (0.1 mmol, 73.1 mg), and potassium phosphate (2.0 mmol, 424.5 mg) were dissolved in 1,4-dioxane (15.0 mL). The reaction system was purged three times with nitrogen, and then reacted for 8 hrs under reflux. After cooling down to room temperature, the reaction mixture was suction filtered, and the solvent in the filtrate was removed by rotary evaporation. The residue was separated by column chromatography (eluant: dichloromethane/methanol (100/1, v/v)) to obtain a pure product as a light yellow solid which was immediate 8b (176.8 mg, yield 40%). NMR test of immediate 8b: (400 MHz, CDCl₃) ¹H NMR (400 MHz, CDCl₃) δ(ppm) 8.38 (s, 1H, Ar—H), 7.93 (s, 1H, Ar—H), 7.66 (d, J=3.3 Hz, 1H, Ar—H), 7.47 (d, J=8.9 Hz, 1H, Ar—H), 6.64 (d, J=8.9 Hz, 1H, Ar—H), 6.56 (s, 1H, Ar—H), 6.54 (d, J=3.3 Hz, 1H, Ar—H), 3.44 (q, J=7.0 Hz, 4H, 2×CH₂), 2.27 (s, 3H, CH₃), 1.69 (s, 9H, 3×CH₃), 1.23 (t, J=6.7 Hz, 6H, 2×CH₃); (151 MHz, CDCl₃) ¹³C NMR (151 MHz, CDCl₃) δ(ppm) 162.1, 155.2, 150.4, 149.3, 147.9, 147.5, 146.7, 131.4, 126.9, 126.2, 126.1, 122.7, 117.9, 109.4, 108.7, 104.7, 97.5, 84.1,44.8, 28.1, 16.5, 12.4.

The immediate 8b (0.3 mmol, 134.2 mg) was dissolved in a mixed solution of concentrated hydrochloric acid (1.0 mL) and 1,4-dioxane (3.0 mL), and stirred for 1.5 hrs at room temperature. The reaction was monitored by TLC. After complete reaction, a saturated sodium bicarbonate solution was added to neutralize the reaction system. The reaction solution was extracted with chloroform (3×30.0 mL). The organic layer was collected, dried over anhydrous Na₂SO₄, and evaporated to remove the solvent. The crude product was separated and purified by column chromatography (eluant: dichloromethane/methanol (30/1, v/v)), to obtain a pure product as a light yellow solid which was dye 1b (97.9 mg, yield 94%). NMR test (400 MHz, DMSO-d₆): ¹H NMR (400 MHz, DMSO-d₆) δ(ppm) 11.73 (s, 1H, N—H), 8.09 (s, 1H, Ar—H), 7.86 (s, 1H, Ar—H), 7.60 (d, J=9.0 Hz, 1H, Ar—H), 7.51 (s, 1H, Ar—H), 6.75 (d, J=8.6 Hz, 1H, Ar—H), 6.58 (s, 1H, Ar—H), 6.48 (s, 1H, Ar—H), 3.46 (q, J=6.9 Hz, 4H, 2×CH₂), 2.22 (s, 3H, CH₃), 1.15 (t, J=6.7 Hz, 6H, 2×CH₃); (151 MHz, DMSO-d₆) ¹³C NMR (151 MHz, DMSO-d₆) δ(ppm) 161.7, 155.1, 150.5, 149.4, 148.0, 144.6, 130.4, 127.2, 126.9, 123.2, 119.5, 118.5, 109.2, 109.1, 100.3, 97.0, 44.4, 16.7, 12.8.

The immediate 6 (1.0 mmol, 357.2 mg), compound 7c (t-butyl5-bromo-1H-pyrazolo[3, 4-b]pyridin-1-carboxylate, 1.2 mmol, 356.4 mg), [1,1′-bis(diphenyl-phosphino) ferrocene]palladium dichloride (0.1 mmol, 73.1 mg), and potassium phosphate (2.0 mmol, 424.5 mg) were dissolved in 1,4-dioxane (15.0 ml). The reaction system was purged three times with nitrogen, and then reacted for 8 hrs under reflux. After cooling down to room temperature, the reaction mixture was suction filtered, and the solvent in the filtrate was removed by rotary evaporation. The residue was separated by column chromatography (eluant: dichloromethane/methanol (100/1, v/v)) to obtain a pure product as a light yellow solid which was immediate 8c (147.9 mg, yield 33%). NMR test of immediate 8c: (400 MHz, CDCl₃) ¹H NMR (400 MHz, CDCl₃) δ(ppm) 8.65 (s, 1H, Ar—H), 8.20 (s, 1H, Ar—H), 8.15 (s, 1H, Ar—H), 7.49 (d, J=9.0 Hz, 1H, Ar—H), 6.67 (d, J=8.5 Hz, 1H, Ar—H), 6.57 (s, 1H, Ar—H), 3.45 (q, J=7.0 Hz, 4H, 2×CH₂), 2.29 (s, 3H, CH₃), 1.75 (s, 9H, 3×CH₃), 1.24 (t, J=7.0 Hz, 6H, 2×CH₃); (151 MHz, CDCl₃,) ¹³C NMR (151 MHz, CDCl₃) δ(ppm) 162.2, 156.1, 152.9, 151.6, 150.5, 150.3, 147.5, 136.3, 132.2, 125.5, 119.3, 115.4, 109.1, 108.8, 108.4, 97.7, 85.9, 44.7, 28.1, 18.4, 12.5.

The immediate 8c (0.3 mmol, 134.5 mg) was dissolved in a mixed solution of concentrated hydrochloric acid (1.0 mL) and 1,4-dioxane (3.0 mL), and stirred for 1.5 hrs at room temperature. The reaction was monitored by TLC. After complete reaction, a saturated sodium bicarbonate solution was added to neutralize the reaction system. The reaction solution was extracted with chloroform (3×30.0 mL). The organic layer was collected, dried over anhydrous Na₂SO₄, and evaporated to remove the solvent. The crude product was separated and purified by column chromatography (eluant: dichloromethane/methanol (30/1, v/v)), to obtain a light yellow solid which was dye 1c (100.3 mg). NMR test: (400 MHz, DMSO-d₆) ¹H NMR (400 MHz, DMSO-d₆) δ(ppm) 13.74 (s, 1H, N—H), 8.42 (s, 1H, Ar—H), 8.18 (s, 1H, Ar—H), 8.16 (s, 1H, Ar—H), 7.62 (d, J=8.6 Hz, 1H, Ar—H), 6.76 (d, J=8.7 Hz, 1H, Ar—H), 6.59 (s, 1H, Ar—H), 3.46 (q, J=6.1 Hz, 4H, 2×CH₂), 2.23 (s, 3H, CH₃), 1.15 (t, J=6.9 Hz, 6H, 2×CH₃); (151 MHz, DMSO-d₆)¹³C NMR (151 MHz, DMSO-d₆)δ(ppm) 161.6, 155.2, 151.2, 151.1, 150.78, 150.1, 133.8, 132.1, 127.3, 124.5, 117.4, 114.4, 109.3, 109.0, 97.0, 44.4, 16.7, 12.8.

The compound 9 (1.0 mmol, 379.2 mg), compound 7a (1.2 mmol, 355.2 mg), [1,1′-bis(diphenylphosphino)ferrocene]palladium dichloride (0.1 mmol, 73.1 mg), and potassium acetate (2.0 mmol, 196.3 mg) were dissolved in 1,4-dioxane (15.0 ml). The reaction system was purged three times with nitrogen, and then reacted for 6 hrs under reflux. After complete reaction, the reaction mixture was cooled down to room temperature, suction filtered, and the solvent in the filtrate was removed by rotary evaporation. The product was separated by column chromatography (eluant: dichloromethane) to obtain immediate 10a as a white solid (300.3 mg, yield 64%). NMR test: (400 MHz, CDCl₃) ¹H NMR (400 MHz, CDCl₃) δ(ppm) 8.66 (t, J=8.0 Hz, 2H, Ar—H),, 8.37 (d, J=8.5 Hz, 1H, Ar—H), 8.29 (s, 1H, Ar—H), 8.21 (d, J=8.5 Hz, 1H, Ar—H), 7.88 (s, 1H, Ar—H), 7.75(d, J=7.6 Hz, 1H, Ar—H), 7.72(d, J=8.8 Hz, 1H, Ar—H), 7.68(d, J=7.8 Hz, 1H, Ar—H), 4.22 (t, J=7.4 Hz, 2H, CH2), 1.78 (s, 9H, 3×CH₃), 1.76-1.71 (m, 2H, CH₂), 1.50-1.44 (m, 2H, CH₂), 1.00 (t, J=7.2 Hz, 3H, CH₃) ; (151 MHz, CDCl₃,) ¹³C NMR (151 MHz, CDCl₃) δ(ppm) 164.2, 164.0, 149.1, 146.0, 139.5, 139.5, 134.4, 132.2, 131.2, 130.8, 130.7, 130.2, 128.7, 128.2, 127.0, 126.1, 123.0, 122.2, 122.1, 114.8, 85.4, 40.3, 30.2, 28.2, 20.4, 13.8.

The immediate 10a (0.5 mmol, 234.6 mg) was dissolved in a mixed solution of concentrated hydrochloric acid (2.0 mL) and 1,4-dioxane (6.0 mL), and stirred overnight at room temperature. The precipitated white solid was suction filtered, and then washed with a saturated sodium bicarbonate solution to obtain a pure product 2a as a white solid (162.4 mg, yield 88%), which was dye 2a. NMR test: (400 MHz, DMSO-d₆) ¹H NMR (400 MHz, DMSO-d6) δ(ppm) 8.56-8.54 (m, 2H, Ar—H), 8.28 (d, J=8.4 Hz, 1H, Ar—H),, 8.22 (s, 1H, Ar—H), 7.94 (s, 1H, Ar—H), 7.82 (t, J=7.3 Hz, 2H, Ar—H), 7.76 (d, J=8.4 Hz, 1H, Ar—H), 7.51 (d, J=8.5 Hz, 1H, Ar—H), 4.07 (t, J=7.3 Hz, 2H,CH₂), 1.67-1.63 (m,2H,CH₂), 1.41-1.35 (m, 2H, CH₂), 0.94 (t, J=7.2 Hz, 3H,CH₃) ; (151 MHz, DMSO-d₆) ¹³C NMR (151MHz, DMSO-d₆) δ(ppm) 163.9, 163.7, 147.2, 140.1, 134.5, 132.9, 131.1, 130.8, 130.8, 130.1, 128.7, 128.4, 127.8, 123.5, 122.8, 122.4, 121.3, 111.0, 40.5, 30.1, 20.2, 14.2.

The compound 9 (1.0 mmol, 379.2 mg), compound 7b (1.2 mmol, 355.2 mg), [1,1′-bis(diphenylphosphino)ferrocene]palladium dichloride (0.1 mmol, 73.1 mg), and potassium acetate (2.0 mmol, 196.3 mg) were dissolved in 1,4-dioxane (15.0 ml). The reaction was refluxed for 6 hrs under nitrogen atmosphere. After the reaction was completed, the reaction mixture was cooled to room temperature and suction filtered to obtain a filtrate. The filtrate was evaporated to remove the solvent. The crude product was separated and purified by column chromatography (eluant: dichloromethane/methanol (100/1, v/v)), to obtain a white solid (243.9 mg, yield 52%). NMR test of immediate 10b: (400 MHz, CDCl₃) ¹H NMR (400 MHz, CDCl₃) δ(ppm) 8.68 (d, J=7.6 Hz, 1H, Ar—H), 8.66-8.64 (m, 2H, Ar—H), 8.22 (d, J=8.4 Hz, 1H, Ar—H), 8.04 (s, 1H, Ar—H), 7.78 (d, J=3.8 Hz, 1H, Ar—H), 7.75 (d, J=7.6Hz, 1H, Ar—H), 7.71 (d, J=8.1 Hz, 1H, Ar—H), 4.23 (t, J=7.5 Hz, 2H, CH₂), 1.79-1.75 (m, 2H, CH₂), 1.72 (s, 9H, 3×CH₃), 1.50-1.45 (m, 2H, CH₂), 1.00 (t, J=7.2 Hz, 3H, CH₃); (151 MHz, CDCl₃,) ¹³C NMR (151 MHz, CDCl₃) δ(ppm) 164.2, 164.0, 148.1, 147.8, 145.7, 143.7, 132.1, 131.3, 130.7, 130.4, 130.1, 129.55, 128.6, 128.5, 127.9, 127.1, 123.0, 122.8, 122.3, 104.5, 84.6, 40.3, 30.2, 28.1, 20.4, 13.8.

The immediate 10b (0.5 mmol, 234.6 mg) was dissolved in a mixed solution of concentrated hydrochloric acid (2.0 ml) and 1,4-dioxane (6.0 ml), and stirred overnight at room temperature to precipitate a solid. The precipitated solid was suction filtered, and then the filter cake was washed with a saturated sodium bicarbonate solution to obtain a white solid (164.3 mg, yield 89%), which was dye 2b. NMR test: (400 MHz, DMSO-d₆)¹H NMR (400 MHz, DMSO-d₆)δ(ppm) 11.95 (s, 1H, N—H), 8.57 (d, J=4.1 Hz, 1H, Ar—H), 8.55 (d, J=3.5 Hz, 1H, Ar—H), 8.37 (s, 1H, Ar—H), 8.31 (d, J=8.4 Hz, 1H, Ar—H), 8.16 (s, 1H, Ar—H), 7.88-7.83 (m, 2H, Ar—H), 7.63 (s, 1H, Ar—H), 6.59 (s, 1H, Ar—H), 4.08 (t, J=7.3 Hz, 2H, CH₂), 1.69-1.61 (m,2H, CH₂), 1.42-1.35 (m, 2H, CH₂), 0.95 (t, J=7.3 Hz, 3H, CH₃); (151 MHz, DMSO-d₆) ¹³C NMR (151 MHz, DMSO-d₆)δ(ppm) 163.8, 163.6, 148.7, 145.0, 143.6, 132.8, 131.2, 130.7, 130.3, 129.7, 129.1, 128.4, 127.9, 127.9, 126.3, 122.8, 121.4, 119.8, 100.8, 40.5, 30.1, 20.2 14.2.

The compound 9 (1.0 mmol, 379.2 mg), compound 7c (1.2 mmol, 356.4 mg), [1,1′-bis(diphenylphosphino)ferrocene]palladium dichloride (0.1 mmol, 73.1 mg), and potassium acetate (2.0 mmol, 196.3 mg) were dissolved in 1,4-dioxane (15.0 ml). The reaction was refluxed for 4 hrs under nitrogen atmosphere. After the reaction was completed, the reaction mixture was cooled to room temperature and suction filtered to obtain a filtrate. The filtrate was rotary evaporated. The crude product was separated and purified by column chromatography (developing agent: dichloromethane/methanol (100/1, v/v)), to obtain a white solid (150.5 mg, yield 32%). NMR test of immediate 10c: (400 MHz, CDCl₃) ¹H NMR (400 MHz, CDCl₃) δ(ppm) 8.91 (s, 1H, Ar—H), 8.70 (d, J =7.6 Hz, 1H, Ar—H), 8.68 (d, J=7.4 Hz, 1H, Ar—H), 8.31 (s, 1H, Ar—H), 8.26 (s, 1H, Ar—H), 8.13 (d, J=8.4Hz, 1H, Ar—H), 7.77-7.73 (m, 2H, Ar—H), 4.23 (t, J=7.5 Hz, 2H, CH₂), 1.78 (s, 9H, 3×CH₃), 1.74-1.71 (m, 2H, CH₂), 1.52-1.45 (m, 2H, CH₂), 1.00 (t, J=7.3 Hz, 3H, CH₃); (151 MHz, CDCl₃,) ¹³C NMR (151 MHz, CDCl₃) δ(ppm) 164.0, 163.8, 151.6, 151.5, 147.7, 142.1, 137.4, 131.4, 131.4, 131.0, 130.6, 130.6, 130.2, 128.7, 127.5, 123.2, 122.9, 117.6, 85.9, 40.4, 30.2, 28.1, 20.4, 13.8.

The immediate 10c (0.3 mmol, 141.1 mg) was dissolved in a mixed solution of concentrated hydrochloric acid (1.0 mL) and 1,4-dioxane (3.0 mL), and stirred overnight at room temperature to precipitate a solid. The precipitated solid was suction filtered, and then the filter cake was washed with a saturated sodium bicarbonate solution to obtain a white solid (119.9 mg, yield 85%), which was dye 2c. NMR test: (400 MHz, DMSO-d₆)¹H NMR (400 MHz, DMSOd6) δ(ppm) 13.94 (s, 1H, N—H), 8.69 (s, 1H, Ar—H), 8.59-8.56 (m, 2H, Ar—H) 8.48 (s, 1H, Ar—H), 8.29-8.27 (m, 2H, Ar—H), 7.92 (d, J=7.3 Hz, 1H, Ar—H), 7.87 (t, J=7.6 Hz, 1H, Ar—H), 4.09 (t, J=6.2 Hz, 2H, CH₂), 1.67-1.64 (m, 2H, CH₂), 1.41-1.35 (m, 2H, CH₂), 0.95 (t, J=6.8 Hz, 3H, CH₃); (151 MHz, DMSO-d₆) ¹³C NMR (151 MHz, DMSO-d₆) δ(ppm) 163.8, 163.6, 151.8, 149.9, 143.8, 134.3, 132.5, 131.6, 131.3, 130.7, 130.2, 129.3, 128.4, 128.1, 127.5, 122.9, 121.9, 114.6, 40.5, 30.1, 20.2, 14.2.

The compound 11 (2.0 mmol, 668.3 mg) was dissolved in an anhydrous tetrahydrofuran solution (30.0 ml), and then N-phenylbis(trifluoromethane-sulfonyl)imide (4.0 mmol, 1.4 g) and triethylamine (4.0 mmol, 0.6 ml). Under a nitrogen atmosphere, the reaction was stirred at room temperature for 24 hrs. After the reaction was completed, the reaction solution was evaporated to remove the solvent under vacuum. The residue was separated by column chromatography (eluant: dichloromethane/methanol (50/1, v/v)) to obtain a green solid (559.3 mg, yield 60%). NMR test of immediate 12: (400 MHz, CDCl₃) ¹H NMR (400 MHz, CDCl₃) δ(ppm) 8.70 (d, J=8.8 Hz, 1H, Ar—H), 8.15 (s, 1H, Ar—H), 7.58 (d, J=5.6 Hz, 1H, Ar—H), 7.55 (d, J=5.8 Hz, 1H, Ar—H), 6.66 (d, J=9.1 Hz, 1H, Ar—H), 6.43 (s, 1H, Ar—H), 6.38 (s, 1H, Ar—H), 3.48 (q, J=7.1 Hz, 4H, 2×CH₂), 1.26 (t, J=7.1 Hz, 6H, 2×CH₃); (151 MHz, DMSO-d₆) ¹³C NMR (151 MHz, DMSO-d₆) δ(ppm) 181.3, 152.3, 151.4, 150.5, 147.0, 137.6, 133.4, 131.7, 131.5, 126.5, 125.2, 124.0, 118.1, 110.3, 105.5, 96.1,45.2, 12.6.

The immediate 12 (2.0 mmol, 932.2 mg), Bis(pinacolato)diboron (2.5 mmol, 634.8 mg), [1,1′-bis(diphenylphosphino)ferrocene]palladium dichloride (0.2 mmol, 146.3 mg), and potassium acetate (4.0 mmol, 392.6 mg) were dissolved in 1,4-dioxane (40.0 ml). The reaction mixture was refluxed for 8 hrs under a nitrogen atmosphere. After the reaction was completed, the reaction solution was cooled to room temperature and suction filtered. The solvent was removed from the filtrate by rotary evaporation. The crude product was separated and purified by column chromatography (eluant: dichloromethane/methanol (100/1, v/v)), to obtain a green solid (790.7 mg, yield 89%). NMR test of immediate 13: (400 MHz, CDCl₃) ¹H NMR (400 MHz, CDCl₃) δ(ppm) 8.78 (s, 1H, Ar—H), 8.61 (d, J=7.6 Hz, 1H, Ar—H), 8.10 (d, J=7.6 Hz, 1H, Ar—H), 7.58 (d, J=8.8 Hz, 1H, Ar—H), 6.63 (d, J=8.5 Hz, 1H, Ar—H), 6.43 (s, 1H, Ar—H), 6.37 (s, 1H, Ar—H), 3.45 (q, J=6.7 Hz, 4H, 2×CH₂), 1.38 (s, 12H, 4×CH₃), 1.25 (t, J =7.1 Hz, 6H, 2×CH₃) ; (151 MHz, CDCl₃) ¹³C NMR (151 MHz, CDCl₃) δ(ppm) 183.8, 152.2, 150.8, 146.7, 139.9, 136.9, 134.1, 132.7, 131.2, 130.8, 125.0, 122.8, 109.7, 105.8, 96.2, 84.1, 83.1,45.1, 24.9, 24.5, 12.6.

The immediate 13 (1.0 mmol, 444.2 mg), compound 7a (1.2 mmol, 355.2 mg), [1,1′-bis(diphenylphosphino)ferrocene]palladium dichloride (0.1 mmol, 73.1 mg), and potassium acetate (2.0 mmol, 196.3 mg) were dissolved in 1,4-dioxane (35.0 ml).

The reaction system was purged 3 times with nitrogen, and then heated for 12 hrs under reflux. After the reaction was completed, the reaction solution was cooled to room temperature and suction filtered. The filtrate was evaporated to remove the solvent. The crude product was separated by column chromatography (eluant: dichloromethane/methanol (100/1, v/v)), to obtain a reddish brown solid (192.3 mg, yield 43%). NMR test of immediate 14a: (400 MHz, CDCl₃) ¹H NMR (400 MHz, CDCl₃) δ(ppm) 8.72 (d, J=8.3 Hz, 1H, Ar—H), 8.59 (s, 1H, Ar—H), 8.28 (d, J =8.9 Hz, 1H, Ar—H), 8.25 (s, 1H, Ar—H), 8.10 (s, 1H, Ar—H), 8.00 (d, J=8.3 Hz, 1H, Ar—H), 7.95 (d, J=8.7 Hz, 1H, Ar—H), 7.63 (d, J=9.0 Hz, 1H, Ar—H), 6.69 (d, J=9.1 Hz, 1H, Ar—H), H), 6.49 (s, 1H, Ar—H), 6.43 (s, 1H, Ar—H), 3.47 (q, J=6.9 Hz, 4H, 2×CH₂), 1.76 (s, 9H, 3×CH₃). 1.27 (t, J=7.0 Hz, 6H, 2×CH₃); (151 MHz, CDCl₃) ¹³C NMR (151 MHz, CDCl₃) δ(ppm) 183.5, 152.2, 150.7, 149.1, 146.7, 141.7, 139.8, 139.4, 139.3, 135.9, 132.1, 131.1, 130.9, 129.8, 128.6, 126.68, 125.1, 124.6, 124.1, 119.5, 114.9, 109.8, 105.7, 96.2, 85.1,45.1, 28.2, 12.6.

The immediate 14a (0.3 mmol, 160.3 mg) was dissolved in a mixed solution of concentrated hydrochloric acid (2.0 mL) and 1,4-dioxane (6.0 mL), and stirred at room temperature. The reaction was monitored by TLC. After complete reaction, a saturated sodium bicarbonate solution was added to neutralize the reaction system. The reaction solution was extracted with chloroform (3×30.0 mL). The organic layer was collected, dried over anhydrous Na₂SO₄, and evaporated to remove the solvent. The crude product was separated and purified by column chromatography (eluant: dichloromethane/methanol (30/1, v/v)), to obtain a dark green solid (121.1 mg, yield 93%), which was dye 3a. NMR test: (400 MHz, DMSO-d₆) ¹H NMR (400 MHz, DMSO-d₆) δ(ppm)13.21 (s, 1H, N—H), 8.57 (d, J=8.3 Hz, 1H, Ar—H), 8.35 (s, 1H, Ar—H), 8.16-8.12 (m, 3H, Ar—H), 7.76 (d, J=8.7 Hz, 1H, Ar—H), 7.63 (d, J=8.7 Hz, 1H, Ar—H), 7.60 (d, J=9.2 Hz, 1H, Ar—H), 6.81 (d, J=8.7 Hz, 1H, Ar—H), 6.64 (s, 1H, Ar—H), 6.29 (s, 1H, Ar—H). 3.51 (q, J =6.9 Hz, 4H, 2×CH₂), 1.17, (t, J=6.8 Hz, 6H, 2×CH3); (151 MHz, DMSO-d6) ¹³C NMR (151 MHz, DMSO-d₆) δ(ppm) 181.8, 151.8, 150.7, 146.3, 142.0, 138.0, 134.2, 131.4, 131.3, 130.8, 129.8, 129.7, 129.5, 125.4, 124.3, 124.2, 122.5, 118.8, 110.8, 110.3, 109.4, 104.5, 96.0, 44.4,12.4.

The immediate 13 (1.0 mmol, 444.2 mg), compound 7b (1.2 mmol, 355.2 mg), [1,1′-bis(diphenylphosphino)ferrocene]palladium dichloride (0.1 mmol, 73.1 mg) and potassium acetate (2.0 mmol, 196.3 mg) were dissolved in 1,4-dioxane (35.0 ml). The reaction was refluxed for 7 hrs under nitrogen atmosphere. After the reaction was completed, the reaction solution was cooled to room temperature and suction filtered. The solvent was removed from the filtrate by rotary evaporation. The residue was separated by column chromatography (eluant: dichloromethane/methanol (100/1, v/v)) to obtain a brown solid (204.1 mg, yield 47%). NMR test of immediate 14b: (400 MHz, CDCl₃) ¹H NMR (400 MHz, CDCl₃) δ(ppm) 8.86 (s, 1H, Ar—H), 8.74 (d, J=8.3 Hz, 1H, Ar—H), 8.57 (s, 1H, Ar—H), 8.24 (s, 1H, Ar—H), 7.98 (d, J=8.2 Hz, 1H, Ar—H), 7.70 (d, J=3.8 Hz, 1H, Ar—H), 7.63 (d, J=9.0 Hz, 1H, Ar—H), 6.68 (d, J=8.9 Hz, 1H, Ar—H), 6.60 (d, J=3.9 Hz, 1H, Ar-—H), 6.43 (s, 1H, Ar—H), 3.47 (q, J=7.0 Hz, 4H, 2×CH2), 1.70 (s, 9H, 3×CH₃), 1.26 (t, J=6.8 Hz, 6H, 2×CH₃); (151 MHz, CDCl₃) ¹³C NMR (151 MHz, CDCl₃) δ(ppm) 183.5, 152.3, 150.8, 148.0, 147.9, 146.8, 144.1, 140.1, 139.5, 132.2, 131.2, 131.1, 131.0, 129.9, 127.7, 127.4, 125.1, 124.7, 124.2, 123.2, 109.8, 105.8, 104.7, 96.3, 84.2, 45.1, 28.1, 12.6.

The immediate 14b (0.3 mmol, 160.3 mg) was dissolved in a mixed solution of concentrated hydrochloric acid (2.0 ml) and 1,4-dioxane (6.0 ml), and stirred at room temperature. The reaction was monitored by TLC. After complete reaction, a saturated sodium bicarbonate solution was added. The reaction solution was extracted with chloroform (3×30.0 ml). The organic layer was collected, dried over anhydrous Na₂SO₄, and evaporated to remove the solvent. The crude product was separated and purified by column chromatography (eluant: dichloromethane/methanol (30/1, v/v)), to obtain a green solid (118.5 mg, yield 91%), which was dye 3b. NMR test: (400 MHz, DMSO-d₆) ¹H NMR (400 MHz, DMSO-d₆) δ(ppm) 11.82 (s, 1H, N—H), 8.66-8.63 (m, 2H, Ar—H), 8.40 (d, J=5.6 Hz, 1H, Ar—H), 8.21 (d, J=7.0 Hz, 1H,Ar—H), 7.67 (d, J=9.0 Hz, 1H, Ar—H), 7.56 (s, 1H, Ar—H), 6.87 (d, J=9.0 Hz, 1H, Ar—H), 6.71 (s, 1H, Ar—H), 6.56 (s, 1H, Ar—H), 6.36 (s, 1H, Ar—H), 3.52 (q, J=7.0 Hz, 4H, 2×CH₂), 1.17 (t, J=6.8 Hz, 6H, 2×CH₃); (151 MHz, TFA-d) ¹³C NMR (151 MHz, TFA-d) δ(ppm) 150.8, 148.0, 138.0, 137.1,136.8,134.3, 131.3, 130.6, 130.5, 130.2, 130.0, 127.6, 125.8, 123.4, 120.0, 115.7, 114.9, 113.7, 113.0, 104.0, 101.9, 50.5, 10.2.

The immediate 13 (1.0 mmol, 444.2 mg), compound 7c (1.2 mmol, 356.4 mg), [1,1′-bis(diphenylphosphino)ferrocene]palladium dichloride (0.1 mmol, 73.1 mg) and potassium acetate (2.0 mmol, 196.3 mg) were dissolved in 1,4-dioxane (35.0 ml). The reaction was refluxed for 7 hrs under nitrogen atmosphere. After the reaction was completed, the reaction solution was cooled to room temperature and suction filtered.

The solvent was removed from the filtrate by rotary evaporation. The residue was separated by column chromatography (eluant: dichloromethane/methanol (100/1, v/v)) to obtain a brown solid (183.8 mg, yield 35%). NMR test of immediate 14c: (400 MHz, CDCl₃) ¹H NMR (400 MHz, CDCl₃) δ(ppm) 9.11 (s, 1H, Ar—H), 8.75 (d, J=7.0 Hz, 1H, Ar—H), 8.56 (s, 1H, Ar—H), 8.41 (s, 1H, Ar—H), 8.26 (s, 1H, Ar—H), 7.96 (d, J=7.9 Hz, 1H, Ar—H), 7.63 (d, J=7.8 Hz, 1H, Ar—H), 6.69 (d, J=8.7 Hz, 1H, Ar—H), 6.48 (s, 1H, Ar—H), 6.43 (s, 1H, Ar—H), 3.48 (q, J=5.8 Hz, 4H, 2×CH₂), 1.77 (s, 9H, 3×CH₃). 1.27 (t, J=5.4 Hz, 6H, 2×CH₃); (151 MHz, CDCl₃) ¹³C NMR (151 MHz, CDCl₃) δ(ppm) 183.2, 152.4, 151.5, 151.0, 150.1, 147.8, 146.9, 139.2, 138.7, 137.6, 132.3, 132.1, 131.6, 131.3, 129.8, 128.4, 125.2, 125.0, 124.4, 118.0, 110.0, 105.7, 96.3, 85.5, 45.1, 28.1, 12.6.

The immediate 14c (0.3 mmol, 166.0 mg) was dissolved in a mixed solution of concentrated hydrochloric acid (2.0 mL) and 1,4-dioxane (6.0 mL), and stirred at room temperature. The reaction was monitored by TLC. After complete reaction, a saturated sodium bicarbonate solution was added. The reaction solution was extracted with chloroform (3×30.0 mL). The organic layer was collected, dried over anhydrous Na₂SO₄, and evaporated to remove the solvent. The crude product was separated by column chromatography (eluant: dichloromethane/methanol (30/1, v/v)), to obtain a purple solid (113.6 mg, yield 87%), which was dye 3c. (400MHz, DMSO-d₆) ¹H NMR (400MHz, DMSO-d₆) δ(ppm) 13.81 (s,1H,N—H), 8.97(s,1H,Ar—H), 8.66-8.64 (m, 2H, Ar—H), 8.43 (s, 1H, Ar—H), 8.24-8.22 (m, 2H, Ar—H),7.66 (d, J=8.9 Hz, 1H, Ar—H), 6.87 (d, J=8.3 Hz, 1H, Ar—H), 6.70 (s,1H,Ar—H), 6.36(s,1H,Ar—H), 3.51 (q, J=6.5Hz, 4H, 2×CH₂), 1.17 (t, J=6.0Hz, 6H, 2×CH₃); (151MHz, TFA-d) 13CNMR(151MHz, TFA-d)δ(ppm)180.9, 150.5, 143.5, 143.2, 142.9, 137.3, 134.9, 134.6, 132.7, 132.1, 131.9, 130.0, 125.9, 123.5, 120.2, 115.5, 114.8, 113.6, 112.9, 49.7, 18.5, 10.4.

The dye 1a, dye 1b, dye 1c, dye 2a, dye 2b, dye 2c, dye 3a, dye 3b, and dye 3c prepared above are neutral fluorescent mitochondrial markers based on nitrogen-containing heterocycles of the present invention.

### Comparative Example

The immediate 13 (1.0 mmol, 444.2 mg), compound 15 (1.2 mmol, 249.6 mg), [1,1′-bis(diphenylphosphino)ferrocene]palladium dichloride (0.1 mmol, 73.1 mg) and potassium acetate (2.0 mmol, 196.3 mg) were dissolved in 1,4-dioxane (35.0 ml). The reaction was refluxed for 4 hrs under a nitrogen atmosphere. After the reaction was completed, the reaction solution was cooled to room temperature and suction filtered. The solvent was removed from the filtrate by rotary evaporation. The residue was separated by column chromatography (eluant: dichloromethane/methanol (100/1, v/v)) to obtain a brown solid (298.9 mg, yield 67%), which was dye 3d. ¹H NMR spectrum (400 MHz, CHCl₃) ¹H NMR (400 MHz, CDCl₃) δ(ppm) 8.90 (s, 1H, Ar—H), 8.86 (s, 1H, Ar—H), 8.78 (d, J=8.2 Hz, 1H, Ar—H), 8.72 (s, 1H, Ar—H), 8.47 (s, 1H, Ar—H), 8.23 (s, 2H, Ar—H), 8.13 (d, J=8.1 Hz, 1H, Ar—H), 7.64 (d, J=8.9 Hz, 1H, Ar—H), 6.69 (d, J=8.8 Hz, 1H, Ar—H) 6.49 (s, 1H, Ar—H), 6.44 (s, 1H, Ar—H), 3.48 (q, J=6.9 Hz, 4H, 2×CH₂), 1.28 (t, J=6.8 Hz, 6H, 2×CH₃); ¹³C NMR spectrum: (151 MHz, CDCl₃) ¹³C NMR (151 MHz, CDCl₃) δ(ppm) 183.4, 152.3 150.9, 146.8, 145.5, 144.9, 143.3, 142.6, 141.6, 140.6, 139.3, 132.2, 131.7, 131.2, 130.0, 129.9, 129.7, 127.3, 125.2, 124.8, 124.6, 109.9, 105.8, 96.3, 45.1, 12.6.

The compound 15 (1.2 mmol, 355.2 mg), [1,1′-bis(diphenylphosphino)-ferrocene]palladium dichloride (0.1 mmol, 73.1 mg) and potassium acetate (2.0 mmol, 196.3 mg) were dissolved in 1,4-dioxane (35.0 ml). The reaction system was purged 3 times with nitrogen, and then heated for 4 hrs under reflux. After the reaction was completed, the reaction solution was cooled to room temperature, and suction filtered. The filtrate was evaporated to remove the solvent. The residue was separated by column chromatography (eluant: dichloromethane/methanol) to obtain an orange solid (118.9 mg, yield 22%). NMR test of immediate 16: (400 MHz, CHCl₃) ¹H NMR (400 MHz, CDCl₃)δ(ppm) 8.29 (d, J=8.5 Hz, 1H, Ar—H), 8.24 (s, 1H, Ar—H), 8.01 (s, 1H, Ar—H), 7.86 (d, J=8.6 Hz, 1H, Ar—H), 7.79 (d, J=7.6 Hz, 2H, Ar—H), 7.39 (d, J=7.6 Hz, 2H, Ar—H), 6.01 (s, 2H, Ar—H), 2.57 (s, 6H, 2×CH₃), 1.76 (s, 9H, 3×CH₃), 1.47 (s, 6H, 2×CH₃); (151 MHz, CDCl₃) ¹³C NMR (151 MHz, CDCl₃) δ(ppm) 155.6, 149.1, 143.0, 141.3, 141.2, 139.7, 139.3 136.0 134.1, 131.4, 128.7, 128.5, 127.8, 126.5, 121.3, 119.2 115.0, 85.1, 28.2, 14.6.

The immediate 16 (0.2 mmol, 108.1 mg) was dissolved in a mixed solution of trifluoroacetic acid (1.0 ml) and dichloromethane (2.0 ml). The reaction solution was stirred for 20 min at room temperature, neutralized with a saturated sodium carbonate solution, and extracted with chloroform (3×30.0 mL). The organic layer was collected, dried over anhydrous Na₂SO₄, and evaporated to remove the solvent. The residue was separated by column chromatography (eluant: dichloromethane/methanol (30/1, v/v)) to obtain an orange solid (20.1 mg), which was dye 4. ¹H NMR spectrum (400 MHz, CHCl₃) 1H NMR (400 MHz, CDCl₃)δ(ppm) iH NMR (400 MHz, DMSO)δ13.12 (s, 1H, Ar—H), 8.11 (d, J=9.9 Hz, 2H, Ar—H), 7.88 (d, J=6.9 Hz, 2H, Ar—H), 7.74 (d, J=7.4 Hz, 2H, Ar—H), 7.61 (d, J =7.8 Hz, 2H, Ar—H), 7.41 (d, J=7.1 Hz, 2H, Ar—H), 6.15 (s, 2H, Ar—H), 2.42 (s, 6H, 2×CH₃), 1.40 (s, 6H, 2×CH₃).

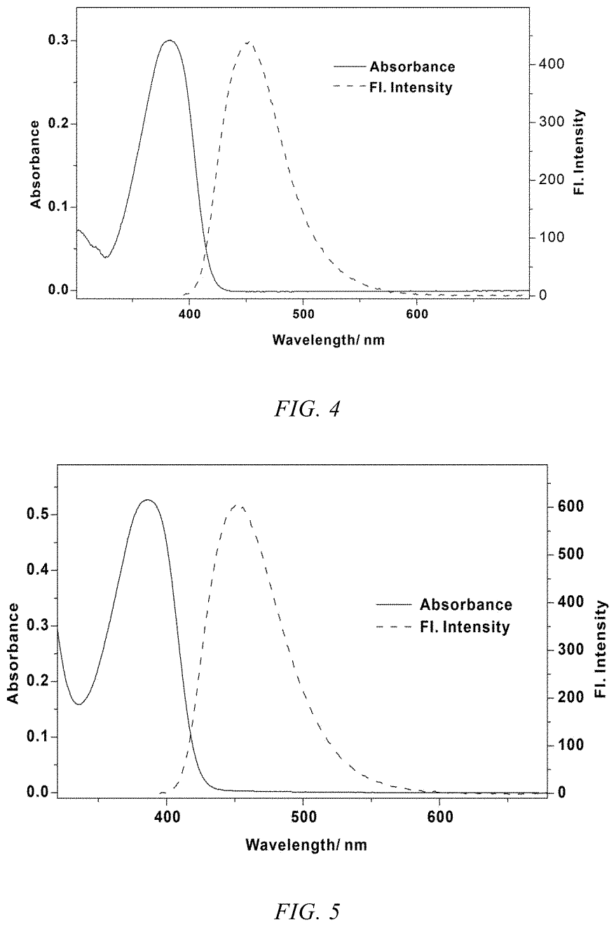

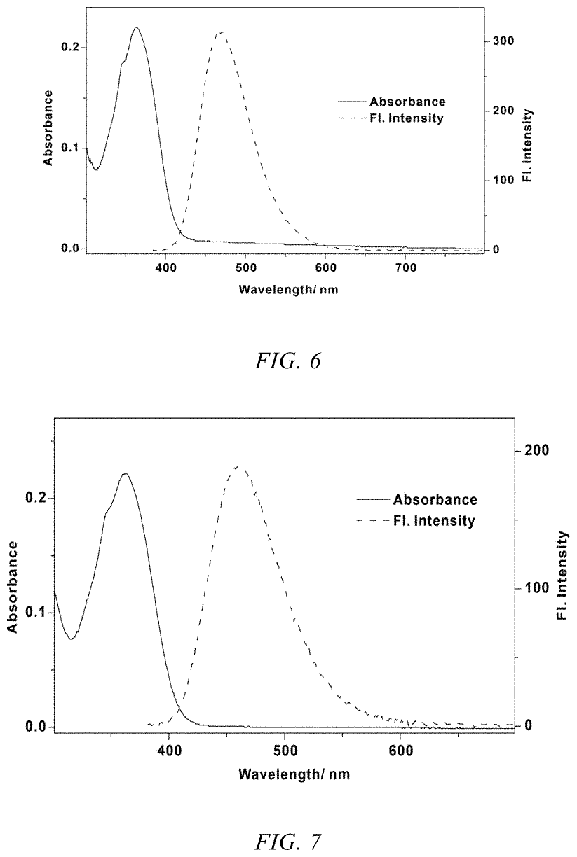

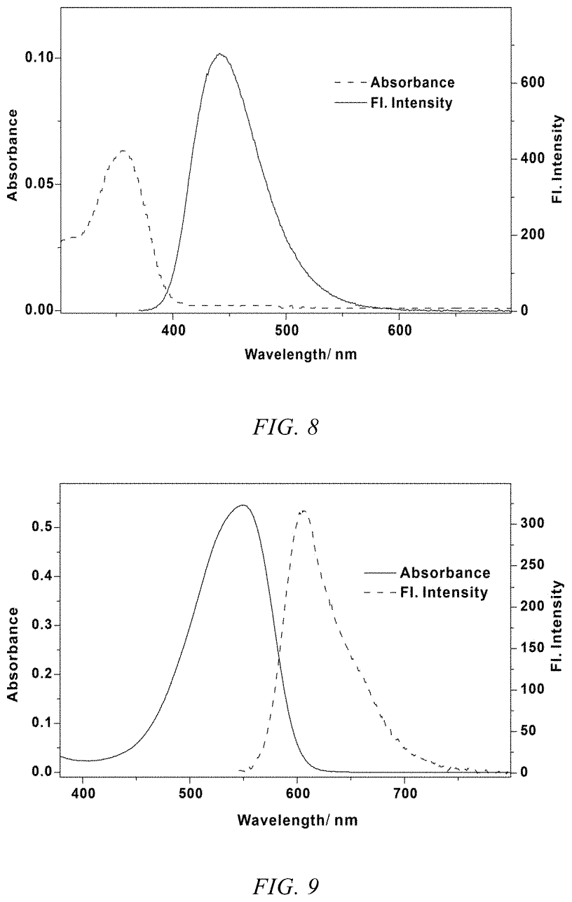

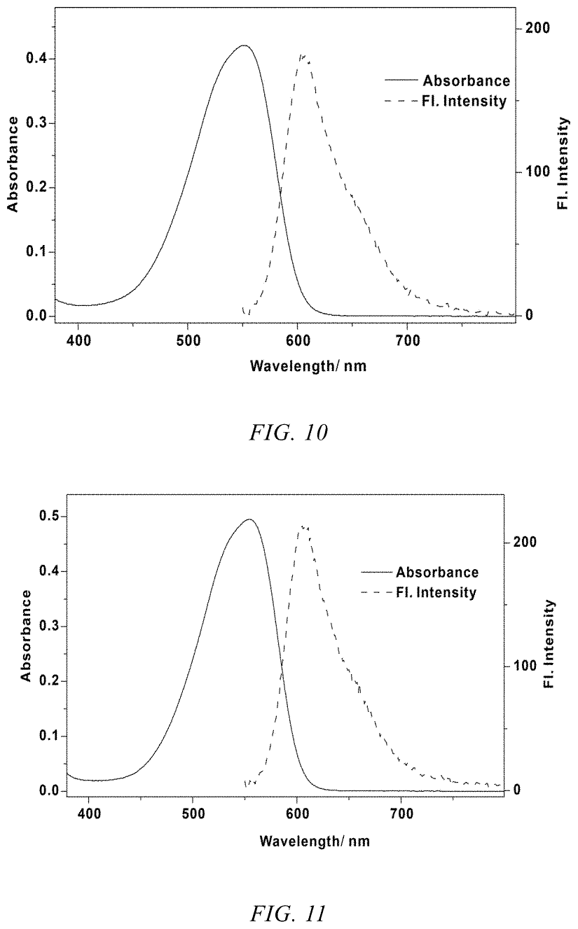

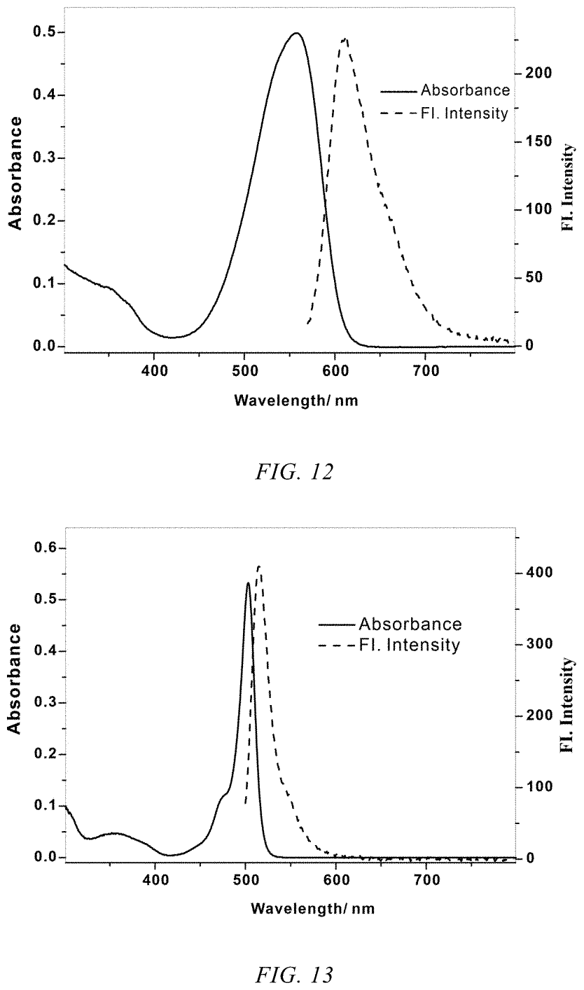

The ultraviolet absorption and fluorescence emission of the dyes prepared in the examples and comparative examples (at a concentration of 10 μM) in chloroform were tested. The horizontal ordinate is the wavelength, and the vertical ordinate is the absorbance and fluorescence intensity, respectively. The results are shown in FIGS. 3 to 13.

In the ultraviolet-visible absorption spectrum, dye 1a has the maximum absorption at 378 nm; and in the fluorescence spectrum, dye 1a has the highest fluorescence intensity at 452 nm, where the excitation wavelength is 370 nm, and the slit width is 3 nm/1.5 nm. In the ultraviolet-visible absorption spectrum, dye 1b has a maximum absorption wavelength of 382 nm; and in the fluorescence spectrum, dye 1b has a maximum emission wavelength of 485 nm, where the excitation wavelength is 374 nm, and the slit width is 3 nm/1.5 nm. In the ultraviolet-visible absorption spectrum, dye 1c has a maximum absorption wavelength of 385 nm; and in the fluorescence spectrum, dye 1c has a maximum emission wavelength of 454 nm, where the excitation wavelength is 380 nm, and the slit width is 3 nm/1.5 nm. In the ultraviolet-visible absorption spectrum, dye 2a has the maximum absorption at 364 nm; and in the fluorescence spectrum, dye 2a has the highest fluorescence intensity at 480 nm, where the excitation wavelength is 374 nm, and the slit width is 3 nm/1.5 nm. In the ultraviolet-visible absorption spectrum, dye 2b has a maximum absorption wavelength of 360 nm; and in the fluorescence spectrum, dye 2b has a maximum emission wavelength of 458 nm, where the excitation wavelength is 370 nm, and the slit width is 3 nm/1.5 nm. In the ultraviolet-visible absorption spectrum, dye 2c has a maximum absorption wavelength of 356 nm; and in the fluorescence spectrum, dye 2c has a maximum emission wavelength of 441 nm, where the excitation wavelength is 360 nm, and the slit width is 3 nm/3 nm. In the ultraviolet-visible absorption spectrum, dye 3a has the maximum absorption at 548 nm; and in the fluorescence spectrum, dye 3a has the highest fluorescence intensity at 606 nm, where the excitation wavelength is 560 nm, and the slit width is 1.5 nm/1.5 nm. In the ultraviolet-visible absorption spectrum, dye 3b has a maximum absorption wavelength of 549 nm; and in the fluorescence spectrum, dye 3b has a maximum emission wavelength of 608 nm, where the excitation wavelength is 540 nm, and the slit width is 1.5 nm/1.5 nm. In the ultraviolet-visible absorption spectrum, dye 3c has a maximum absorption wavelength of 554 nm; and in the fluorescence spectrum, dye 3c has a maximum emission wavelength of 611 nm, where the excitation wavelength is 540 nm, and the slit width is 1.5 nm/1.5 nm. In the ultraviolet-visible absorption spectrum, dye 3d has a maximum absorption wavelength of 556 nm; and in the fluorescence spectrum, dye 3d has a maximum emission wavelength of 619 nm, where the excitation wavelength is 570 nm, and the slit width is 1.5 nm/1.5 nm. In the ultraviolet-visible absorption spectrum, dye 4 has a maximum absorption wavelength of 501 nm; and in the fluorescence spectrum, dye 4 has a maximum emission wavelength of 515 nm, where the excitation wavelength is 495 nm, and the slit width is 1.5 nm/1.5 nm. The above UV absorption and fluorescence emission test methods are conventional methods.

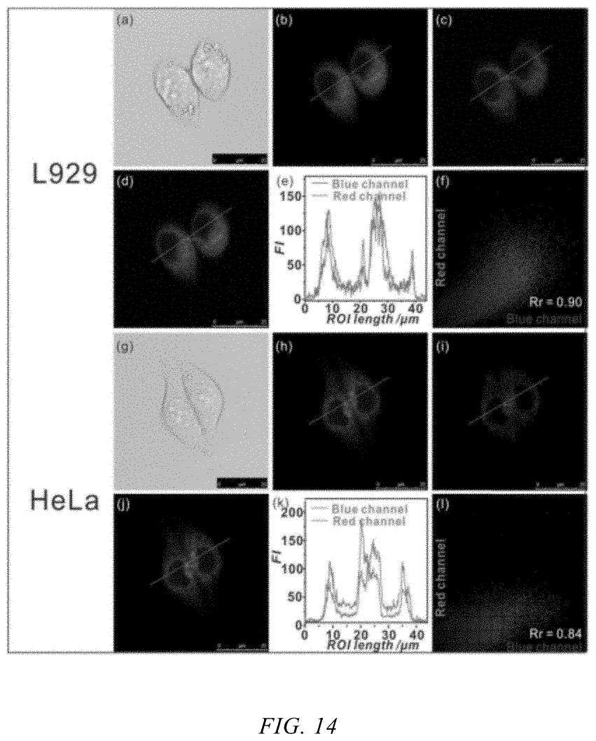

Dye 1a was formulated into a mother liquor in DMSO (dimethyl sulfoxide), and then added to a conventional cell culture medium to give a concentration of dye 1a in the cell culture medium of 1 μM. L929 cells and HeLa cells were respectively co-cultured for 10 min in an incubator at saturated humidity, 37° C., and 5% CO2 (the experiment was same below). The existing red mitochondrial marker Mito Tracker® Red CMXRos (100 nm) was added, and the cells were co-incubated for another 10 min, washed three times with a PBS buffer, and imaged under a laser confocal microscope. In the blue channel, light of 405 nm was used for excitation, and a fluorescence signal in the range of 410-500 nm was collected; and in the red channel, light of 561 nm was used for excitation, and a fluorescence signal in the range of 570-750 nm was collected. The results show that dye 1a has mitochondria labeling ability in both normal cells and cancer cells, and can be used as a blue mitochondrial marker. The results are shown in FIG. 14, where (a) and (g) are the bright-field images; (b) and (h) are the cell images with dye 1a; (c) and (i) are the cell images with the red mitochondrial marker; (d) and (j) are overlapped images with blue channel and red channel, (e) and (k) show the fluorescence intensity of the ROI line in the overlapped images; and (f) and (1) show colocalization assays, with a colocalization coefficient of 0.90 (L929) and 0.84 (HeLa) respectively.

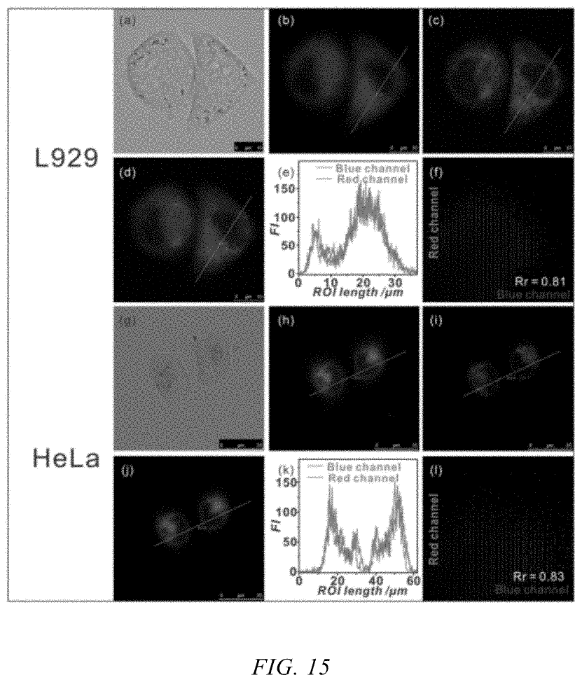

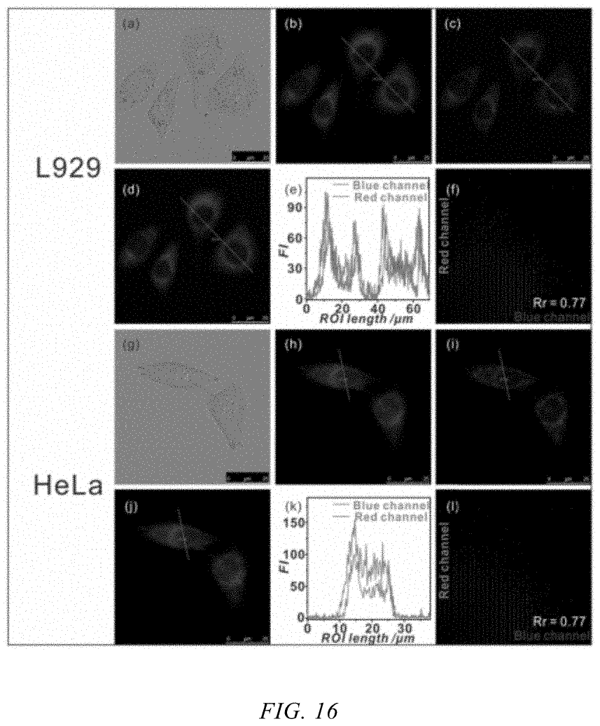

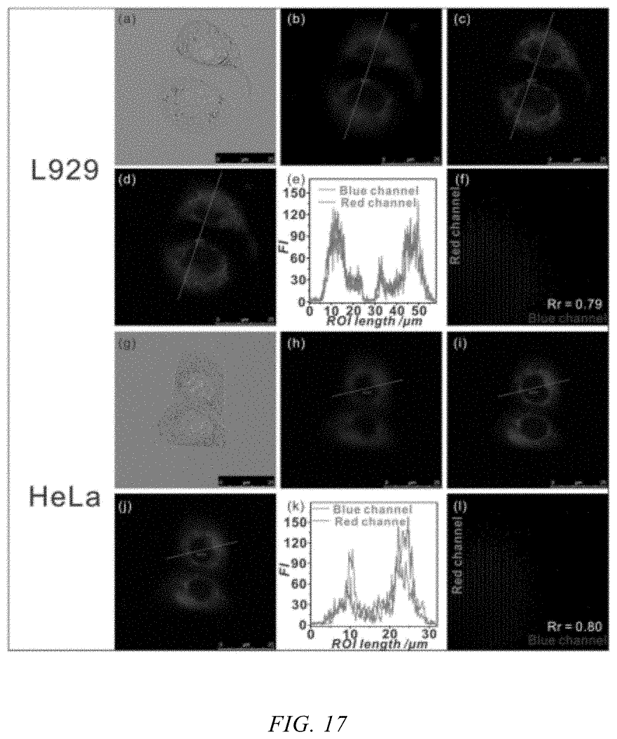

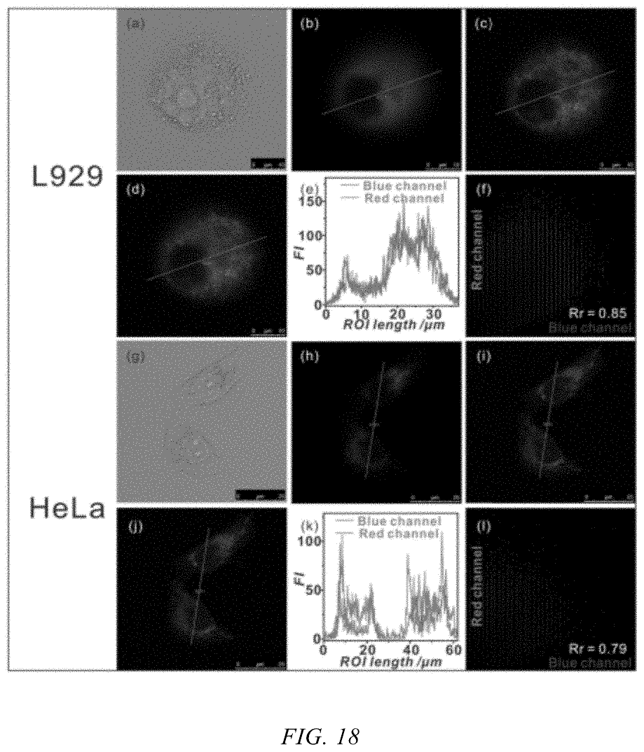

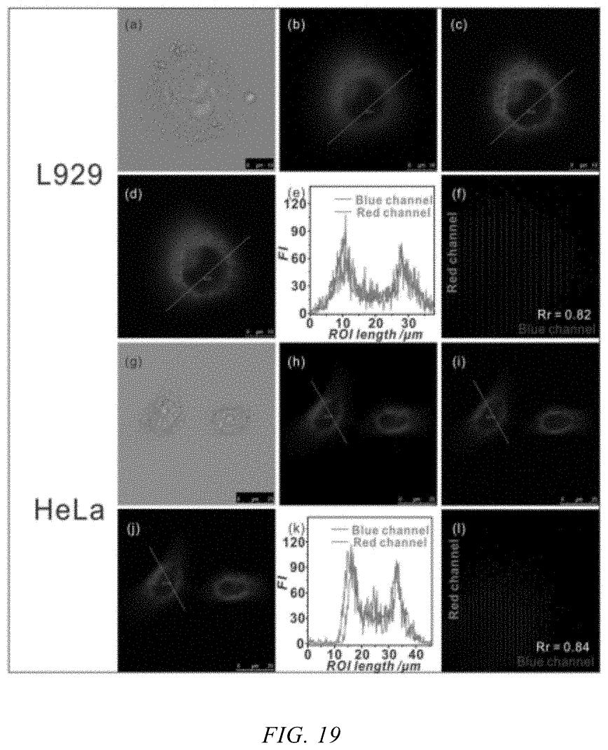

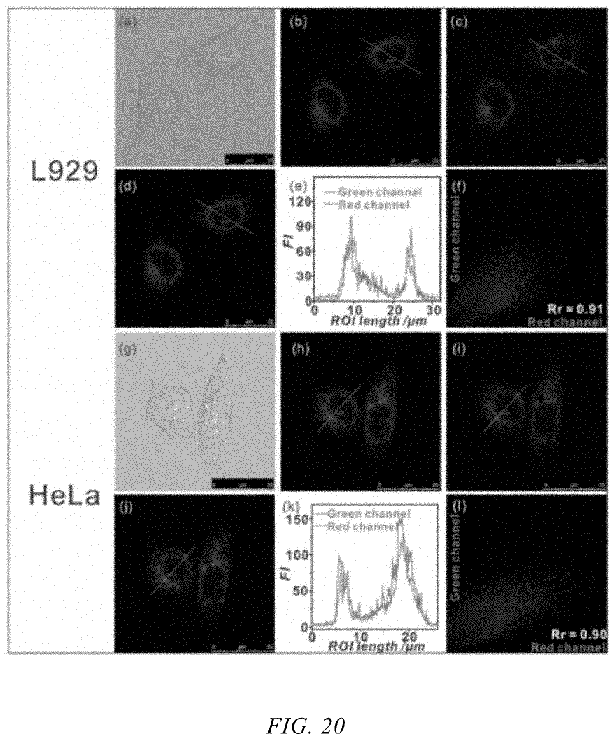

The experiment method with dye 1b, dye 1c, dye 2a, dye 2b, and dye 2c (1 μM) is the same as that with dye 1a above, except that dye 1a is replaced. In FIG. 15, (a) and (g) are the bright-field images; (b) and (h) are the cell images with dye 1b; (c) and (i) are the cell images with the red mitochondrial marker; (d) and (j) are overlapped images with blue channel and red channel, (e) and (k) show the fluorescence intensity of the ROI line in the overlapped images; and (f) and (1) show colocalization assays, with a colocalization coefficient of 0.81 (L929) and 0.83 (HeLa) respectively. In FIG. 16, (a) and (g) are the bright-field images; (b) and (h) are the cell images with dye 1c; (c) and (i) are the cell images with the red mitochondrial marker; (d) and (j) are overlapped images with blue channel and red channel, (e) and (k) show the fluorescence intensity of the ROI line in the overlapped images; and (f) and (1) show a colocalization assay, both having a colocalization coefficient of 0.77. In FIG. 17, (a) and (g) are the bright-field images; (b) and (h) are the cell images with dye 2a; (c) and (i) are the cell images with the red mitochondrial marker; (d) and (j) are overlapped images with blue channel and red channel, (e) and (k) show the fluorescence intensity of the ROI line in the overlapped images; and (f) and (1) show colocalization assays, with a colocalization coefficient of 0.79 (L929) and 0.80 (HeLa) respectively. In FIG. 18, (a) and (g) are the bright-field images; (b) and (h) are the cell images with dye 2b; (c) and (i) are the cell images with the red mitochondrial marker; (d) and (j) are overlapped images with blue channel and red channel, (e) and (k) show the fluorescence intensity of the ROI line in the overlapped images; and (f) and (1) show colocalization assays, with a colocalization coefficient of 0.85 (L929) and 0.79 (HeLa) respectively. In FIG. 19, (a) and (g) are the bright-field images; (b) and (h) are the cell images with dye 2c; (c) and (i) are the cell images with the red mitochondrial marker; (d) and (j) are overlapped images with blue channel and red channel, (e) and (k) show the fluorescence intensity of the ROI line in the overlapped images; and (f) and (1) show colocalization assays, with a colocalization coefficient of 0.82 (L929) and 0.84 (HeLa) respectively. The results show that dye 1b, dye 1c, dye 2a, dye 2b, and dye 2c have mitochondria labeling ability in both normal cells and cancer cells, and can be used as a blue mitochondrial marker.

Dye 3a was formulated into a mother liquor in DMSO (dimethyl sulfoxide), and then added to a conventional cell culture medium to give a concentration of dye 3a in the cell culture medium of 1 μM. L929 cells and HeLa cells were respectively co-cultured for 10 min in an incubator at saturated humidity, 37° C., and 5% CO₂ (the experiment was same below). The existing green mitochondrial marker Mito Tracker® Green FM (100 nm) was added, and the cells were co-incubated for another 10 min, washed three times with a PBS buffer, and imaged under a laser confocal microscope. In the red channel, light of 561 nm was used for excitation, and a fluorescence signal in the range of 570-750 nm was collected. In the green channel, light of 488 nm was used for excitation, and a fluorescence signal in the range of 500-550 nm was collected. The results show that dye 3a has mitochondria labeling ability in both normal cells and cancer cells, and can be used as a red mitochondrial marker. The results are shown in FIG. 20, where (a) and (g) are the bright-field images; (b) and (h) are the cell images with dye 3a; (c) and (i) are the cell images with the green mitochondrial marker; (d) and (j) are overlapped images with red channel and green channel, (e) and (k) show the fluorescence intensity of the ROI line in the overlapped images; and (f) and (1) show colocalization assays, with a colocalization coefficient of 0.91 (L929) and 0.90 (HeLa) respectively.

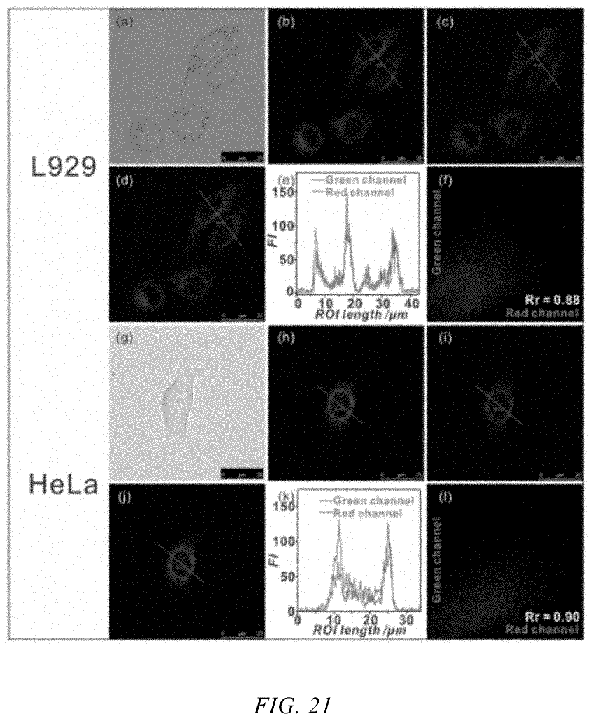

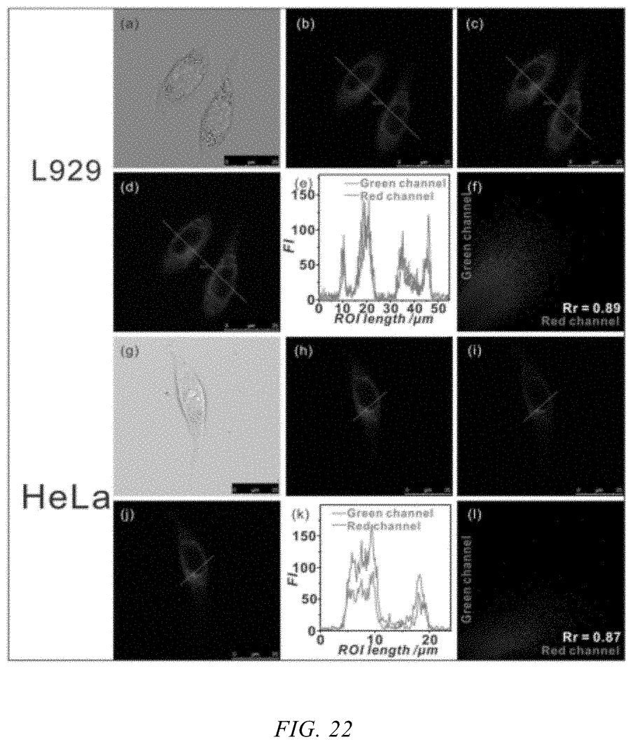

The experiment method with dye 3b (1 μM) and dye 3c (1 μM) is the same as that with dye 3a above, except that dye 3a is replaced. The results show that dye 3b and dye 3c have mitochondria labeling ability in both normal cells and cancer cells, and can be used as a red mitochondrial marker. In FIG. 21, (a) and (g) are the bright-field images; (b) and (h) are the cell images with dye 3b; (c) and (i) are the cell images with the green mitochondrial marker; (d) and (j) are overlapped images with red channel and green channel, (e) and (k) show the fluorescence intensity of the ROI line in the overlapped images; and (f) and (1) show colocalization assays, with a colocalization coefficient of 0.88 (L929) and 0.90 (HeLa) respectively. In FIG. 22, (a) and (g) are the bright-field images; (b) and (h) are the cell images with dye 3c; (c) and (i) are the cell images with the green mitochondrial marker; (d) and (j) are overlapped images with red channel and green channel, (e) and (k) show the fluorescence intensity of the ROI line in the overlapped images; and (f) and (1) show colocalization assays, with a colocalization coefficient of 0.89 (L929) and 0.87 (HeLa) respectively.

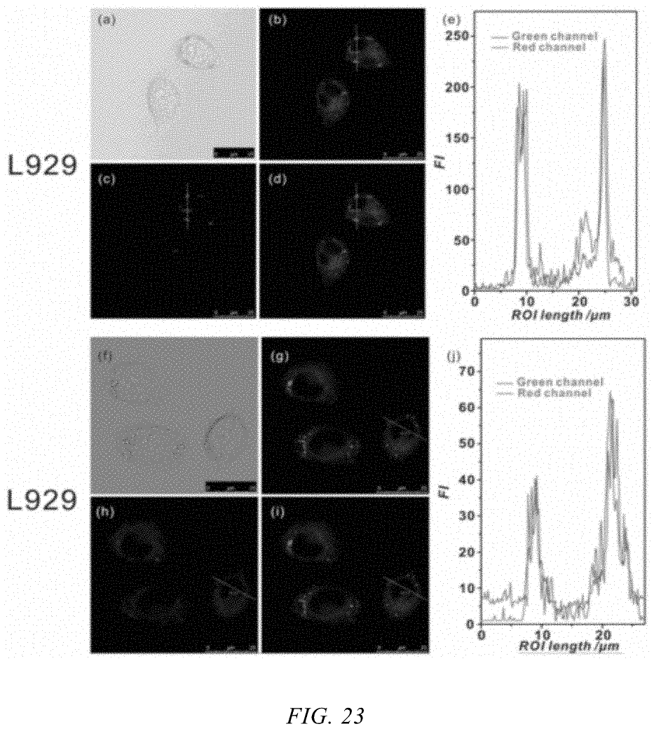

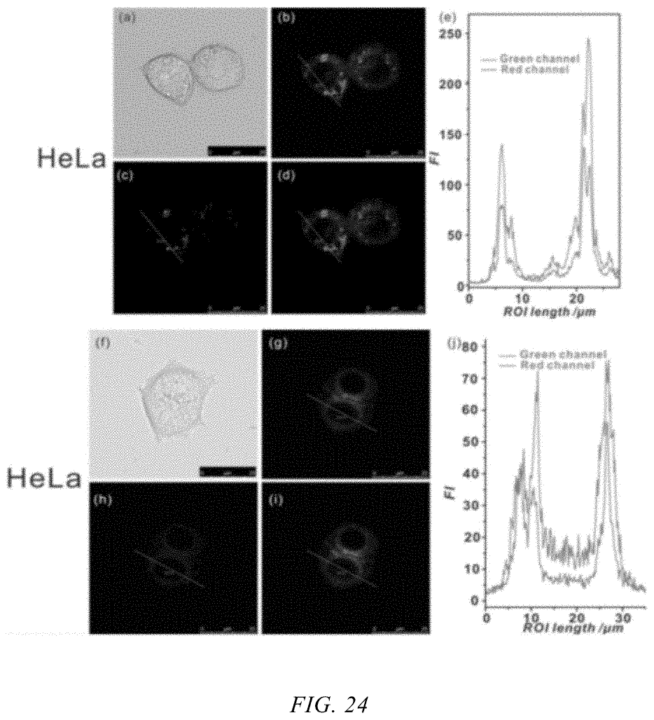

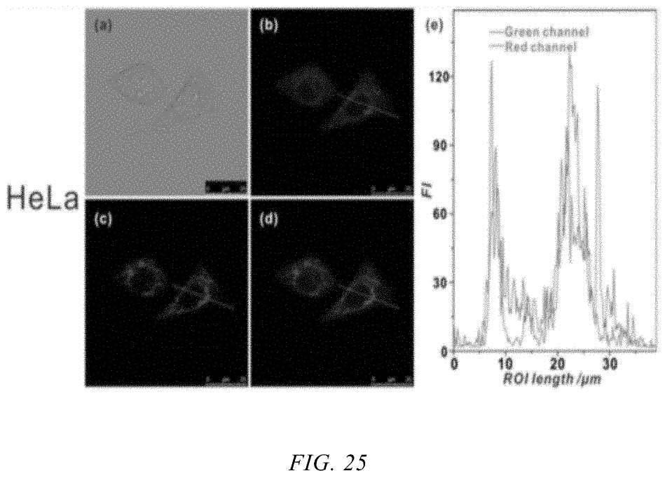

Dye 3d was formulated into a mother liquor in DMSO (dimethyl sulfoxide), and then added to a conventional cell culture medium to give a concentration of dye 3d in the cell culture medium of 1 μM. L929 cells and HeLa cells were respectively co-cultured for 10 min in an incubator at saturated humidity, 37° C., and 5% CO₂. Then the green mitochondrial marker Mito Tracker® Green FM (100 nm) and a green lipid droplet marker were added (the synthesis is as described in CHen, Y.; Wei, X. R.; Sun, R.; Xu, Y. J.; Ge, J. F. Org Biomol CHem 2018, 16, 7619.). The cells were co-incubated for another 10 min, washed three times with a PBS buffer, and imaged under a laser confocal microscope. In the red channel, light of 561 nm was used for excitation, and a fluorescence signal in the range of 570-750 nm was collected. In the green channel, light of 488 nm was used for excitation, and a fluorescence signal in the range of 500-550 nm was collected. The results show that dye 3d simultaneously mark two organelles, i.e. mitochondria and lipid droplets, and is not suitable for use as a mitochondrial marker for cell imaging. The results are shown in FIG. 23, where (a) and (f) are the bright-field images, (b) and (g) are the cell images with dye 3d, (c) is the cell image with the green lipid droplet marker, (h) is the cell image with the green mitochondrial marker, (d) and (i) are overlapped images with red channel and green channel, and (e) and (j) show the fluorescence intensity of the ROI line in the overlapped images. The results are shown in FIG. 24, where (a) and (f) are the bright-field images, (b) and (g) are the cell images with dye 3d, (c) is the cell image with the green lipid droplet marker, (h) is the cell image with the green mitochondrial marker, (d) and (i) are overlapped images with red channel and green channel, and (e) and (j) show the fluorescence intensity of the ROI line in the overlapped images.

Dye 4 was formulated into a mother liquor in DMSO, and then added to a conventional cell culture medium to give a concentration of dye 4 in the cell culture medium of 1 μM. HeLa cells were co-cultured for 10 min in an incubator at saturated humidity, 37° C., and 5% CO₂. The red mitochondrial marker Mito Tracker® Red CMXRos (100 nm) was added, and the cells were co-incubated for another 10 min, washed three times with a PBS buffer, and imaged under a laser confocal microscope. In the red channel, light of 561 nm was used for excitation, and a fluorescence signal in the range of 570-750 nm was collected. In the green channel, light of 488 nm was used for excitation, and a fluorescence signal in the range of 500-550 nm was collected. The results show that dye 4 cannot be overlapped well with the red mitochondrial marker, and cannot be used as a mitochondrial marker for cell imaging. The results are shown in FIG. 25, where (a) is the bright-field image, (b) is the cell image with dye 4, (c) is the cell image with the red mitochondrial marker, (d) is an overlapped image with red channel and green channel, and (e) shows the fluorescence intensity of the ROI line in the overlapped image.

The conventional CCK-8 method was used to test the cytotoxicity of the dyes prepared in the examples, the test time was 6 hrs, and the Meilun CCK-8 cell proliferation and toxicity detection kit was used. The results show that when the dye concentration is 2 μM to 10 μM (where DMSO is the solvent), the survival rates of L929 cells and HeLa cells are both greater than 95%.

What is claimed is:

1. A neutral fluorescent mitochondrial marker based on a nitrogen-containing heterocycle, having a chemical formula of: 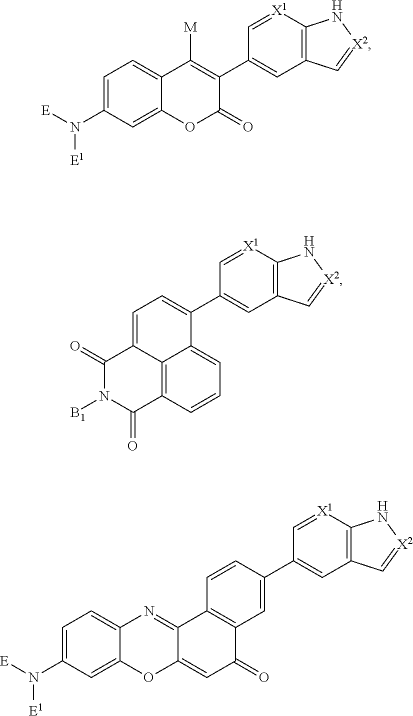
wherein X¹ and X² are independently selected from CH or heteroatoms; and M, E, E¹, and B₁ are independently selected from an alkyl group with less than 6 carbon atoms.

2. The neutral fluorescent mitochondrial marker based on a nitrogen-containing heterocycle according to claim 1, wherein the neutral fluorescent mitochondrial marker has a chemical formula of: 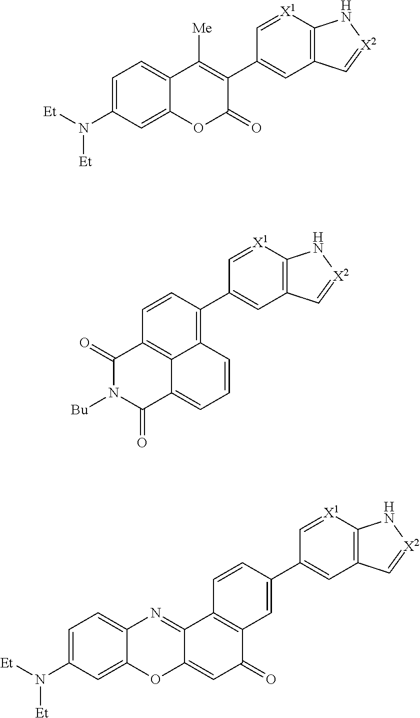
wherein X¹ is selected from CH or N; and X² is selected from CH or N.

3. Use of the neutral fluorescent mitochondrial marker based on a nitrogen-containing heterocycle according to claim 1 in fluorescent labeling of mitochondria; or use of the neutral fluorescent mitochondrial marker based on a nitrogen-containing heterocycle according to claim 1 in the preparation of a fluorescent mitochondrial labeling reagent.

4. A method for preparing a neutral fluorescent mitochondrial marker based on a nitrogen-containing heterocycle according to claim 1, comprising a step of:
(1) reacting a compound 6 with a compound 7 to obtain a compound 8, and deprotecting the compound 8, to obtain a neutral fluorescent mitochondrial marker based on a nitrogen-containing heterocycle; or
(2) reacting a compound 9 with a compound 7 to obtain a compound 10, and deprotecting the compound 10, to obtain a neutral fluorescent mitochondrial marker based on a nitrogen-containing heterocycle; or
(3) reacting a compound 13 with a compound 7 to obtain a compound 14, and deprotecting the compound 14, to obtain a neutral fluorescent mitochondrial marker based on a nitrogen-containing heterocycle.

5. The method for preparing a neutral fluorescent mitochondrial marker based on a nitrogen-containing heterocycle according to claim 4, wherein the deprotection is carried out in the presence of hydrochloric acid; the compound 6 is reacted with the compound in the presence of a noble metal salt catalyst; the compound 9 is reacted with the compound 7 in the presence of a noble metal salt catalyst; and the compound 13 is reacted with the compound 7 in the presence of a noble metal salt catalyst.

6. The method for preparing a neutral fluorescent mitochondrial marker based on a nitrogen-containing heterocycle according to claim 5, wherein the noble metal salt catalyst comprises a palladium salt catalyst.

7. A cell imaging method, comprising steps of:
(1) reacting a compound 6 with a compound 7 to obtain a compound 8, and deprotecting the compound 8, to obtain a neutral fluorescent mitochondrial marker based on a nitrogen-containing heterocycle; or
(2) reacting a compound 9 with a compound 7 to obtain a compound 10, and deprotecting the compound 10, to obtain a neutral fluorescent mitochondrial marker based on a nitrogen-containing heterocycle;
(3) reacting a compound 13 with a compound 7 to obtain a compound 14, and deprotecting the compound 14, to obtain a neutral fluorescent mitochondrial marker based on a nitrogen-containing heterocycle; and
(4) co-incubating the neutral fluorescent mitochondrial marker based on a nitrogen-containing heterocycle prepared in Step (1) or Step (2) with the cells, adding a red mitochondrial marker and imaging the cells after continuous incubation; or
co-incubating the neutral fluorescent mitochondrial marker based on a nitrogen-containing heterocycle prepared in Step (3) with the cells, adding a green mitochondrial marker and imaging the cells after continuous incubation.

8. The cell imaging method according to claim 7, wherein the deprotection is carried out in the presence of hydrochloric acid; the compound 6 is reacted with the compound 7 in the presence of a noble metal salt catalyst; the compound 9 is reacted with the compound 7 in the presence of a noble metal salt catalyst; and the compound 13 is reacted with the compound 7 in the presence of a noble metal salt catalyst.

9. The cell imaging method according to claim 7, wherein the cells are imaged under a laser confocal microscope; in the blue channel, light of 405 nm is used for excitation, and a fluorescence signal in the range of 410-500 nm is collected; in the red channel, light of 561 nm is used for excitation, and a fluorescence signal in the range of 570-750 nm is collected; and in the green channel, light of 488 is used for excitation, and a fluorescence signal in the range of 500-550 nm is collected.

10. The cell imaging method according to claim 7, wherein the cells comprise normal cells and cancer cells.
